(19) United States
(12) Patent Application Publication
(10) Pub. No.: US 2006/0105969 A1
(43) Pub. Date: May 18, 2006

(54) Anthelmintic compositions

(76) Inventor: Christopher Ian Bruce, Sandwich (GB)

Correspondence Address: WARNER-LAMBERT COMPANY, 2800 PLYMOUTH RD, ANN ARBOR, MI, 48105, US

(21) Appl. No.: 11/318,107

(22) Filed: Dec. 23, 2005

Related U.S. Application Data
(63) Continuation of application No. 10/434,136, filed on May 8, 2003, now abandoned.

(30) Foreign Application Priority Data
Jul. 8, 1999 (GB) 9916052.5

(51) Int. Cl. A61K 31/7048; A61K 31/4152

(52) U.S. Cl. 514/28; 514/406

(57) ABSTRACT

Combinations of avermectins and milbemycins and their derivatives with bis-aryl compounds and their use in combating parasite, especially helminth, infections.

This invention relates to combinations, especially synergistic combinations of avermectins and milbemycins and their derivatives with bis-aryl compounds and their use in combating parasite, especially helminth, infections.

The avermectins are a group of broad spectrum antiparasitic agents referred to previously as the C-076 compounds. They are produced by fermenting a strain of the micro-organism Streptomyces avermitilis under aerobic conditions in an aqueous nutrient medium containing inorganic salts and assimilable sources of carbon and nitrogen. The isolation and the chemical structure of the eight individual components which make up the C-076 complex is described in detail in British Patent Specification 1573955.

The C-076 complex comprises eight distinct but closely related compounds described as C-076 A1a, A1b, A2a, A2b, B1a, B1b, B2a and B2b. The “a” series of compounds refers to the natural avermectins wherein the 25-substituent is (S)-sec-butyl and the “b” series to those wherein the 25-substituent is isopropyl. The designations “A” and “B” refer to avermectins wherein the 5-substituent is methoxy or hydroxy, respectively, and the numeral “1” refers to avermectins wherein a double bond is present at the 22-23 position, and numeral “2” to avermectins wherein there is a single bond between C-22 and C-23 and wherein there is a 23-hydroxy substituent.

In our European Patent Applications 0214731, 0284176, 0317148, 0308145, 0340832, 0335541 and 0350187 there are described preparations of compounds related to the naturally-occurring avermectins but having a group at the 25-position other than the isopropyl or (S)-sec-butyl groups found in the original avermectin compounds disclosed in British Patent Specification 1573955. Such compounds may be prepared by fermentation of particular strains of Streptomyces avermitilis in the presence of organic acids or derivatives thereof. Production of such avermectins is described in Journal of Antibiotics (1991), 44, No. 3, pp 357-365.

The milbemycins form another group of related macrolides which are distinguished from the avermectins in lacking a sugar residue attached at the C-13 position. Examples of such compounds are described in UK patent 1390336, and European patent publications 170006, 254583, 334484 and 410615. In addition to these fermentation products a large number of publications describe compounds derived semisynthetically from them, many of which possess useful antiparasitic activities. Some of this chemistry is reviewed in Macrolide Antibiotics, Omura S., Ed., Academic press, New York (1984) and by Davies, H. G., Green, R. H. in Natural product Reports (1986), 3, 87-121 and in Chem. Soc. Rev., 1991, 20, 271-339. It is known that the avermectins and milbemycins and their derivatives are active as antiparasitic agents.

Other avermectin and milbemycin derivatives which may be mentioned are disclosed in the following patents, applications, and their equivalents: European—214 731, 340 932, 334 848, 335 541, 350 187, 410 615 623 137, 712 411, 629 202, 702 688, 674 649, 710 242, 677 054 and 745 089; WO 94/15944, and U.S. Pat. No. 4,199,569.

The terms avermectins and milbemycins used herein includes both naturally occurring compounds and synthetic derivatives thereof, especially those mentioned in the art cited herein.

Bis-aryl compounds having insecticidal and acaricidal properties are also known. These compounds comprise directly joined aromatic rings which may include at least one nitrogen atom. Examples of this class of compounds include N-phenyl pyrazole derivatives having arthropodicidal, plant nematodicidal anthelmintic and anti-protozoal properties, described in EP-0350311A, EP-293117A and in other published patent documents; N-(pyrid-2-yl) pyrazoles described in EP-500209A, EP-511845A and U.S. Pat. No. 5,321,040; 2-phenylimidazoles described in EP-283173A and EP-398499A; N-phenylimidazoles (EP-396427A, U.S. Pat. Nos. 5,180,732 and 5,153,215); N-phenylpyrroles (EP-372982A); 1-phenyl-1,2,3 triazoles and their pyridyl and pyrimidinyl analogues (EP400842A and EP-398499A); N-phenyl-1,2,4-triazoles (EP-398499A and JP 63290870A); 3-phenyl-1,2,4-oxadiazoles and thiadiazoles (JP-06100550A); N-phenylpyridones and thiones (EP 216541A, EP 259048A, EP 367410A and EP 398499A); N-(2-pyridyl) pyridones and thiones (EP 272824A, EP 431468A, JP 03178964A); N-phenylpyridimidinones and thiones, and their 2-pyridyl analogues (EP 338686A, EP 396250A, EP 481604A, AU 9063650A, EP 398499A and EP 357201A); and N-phenyl and N-(2-pyridyl) indoles (JP 06092935A).

Other bis-aryl compounds having such properties which may be mentioned are those disclosed in the following patents, applications and their equivalents: WO 97/07102, EP 0 846 686, WO 98/04530, WO 98/24767, EP 0 933 363, EP 0 957094 and EP 0 959 094.

It has now been found that certain avermectins and milbemycins display unexpected synergy with members of the above-mentioned bis-aryl compounds with respect to their antiparasitic activity, allowing more effective control of parasites, especially those affecting livestock and companion animals. Examples of such parasites include heartworm and various ectoparasites in companion animals. This is particularly unexpected as impairment of biological performance would be anticipated for such a combination, given the reported mode of action of avermectin and milbemycin compounds as agonists at the invertebrate glutamate gated chloride channel-receptor complex and the antagonist activity of the bis-aryl compounds at this site.

According to one aspect of the invention, there is provided a composition comprising a combination of a bis-aryl compound and an avermectin or milbemycin or derivative thereof, and if necessary a suitable pharmaceutical or veterinary carrier.

The invention also includes the use of such a composition in medicine, for instance for treating or preventing parasitic infestations in humans or animals, including infestation by heartworm and other parasites in companion animals such as dogs and cats.

Another aspect of the invention is the administration to an animal of a bis-aryl compound and an avermectin or milbemycin either in combination in the same composition, or via separate treatments, either substantially simultaneously or at spaced intervals, for treating or preventing a parasitic infestation. Different modes of administration can be envisaged by the skilled person where the bis-aryl compound is administered separately from the avermectin or milbemycin compound.

Another aspect to the invention is the use of a composition comprising an avermectin or milbemycin and a bis-aryl compound in the manufacture of an antiparasitic medicament.

Another aspect of the invention is a pharmaceutical pack comprising a bis-aryl compound and an avermectin or milbemycin.

For use in mammals, including humans, the compounds, either alone or in the combinations mentioned above, can be administered alone, but will generally be administered in admixture with a pharmaceutically or veterinarily acceptable diluent or carrier selected with regard to the intended route of administration and standard pharmaceutical practice. For example, they can be administered orally, including sublingually, in the form of tablets containing such excipients as starch or lactose, or in capsules or ovules either alone or in admixture with excipients, or in the form of elixirs, solutions or suspensions containing flavouring or colouring agents. The compounds could be incorporated into capsules or tablets for targetting the colon or duodenum via delayed dissolution of said capsules or tablets for a particular time following oral administration. Dissolution could be controlled by susceptibility of the formulation to bacteria found in the duodenum or colon, so that no substantial dissolution takes places before reaching the target area of the gastrointestinal tract. The compounds can be injected parenterally, for example, intravenously, intramuscularly or subcutaneously. For parenteral administration, they are best used in the form of a sterile aqueous solution or suspension which may contain other substances, for example, enough salt or glucose to make the solution isotonic with blood. They can be administered topically, in the form of sterile creams, gels, suspensions, lotions, ointments, dusting powders, sprays, drug-incorporated dressings or via a skin patch. For example they can be incorporated into a cream consisting of an aqueous or oily emulsion of polyethylene glycols or liquid paraffin, or they can be incorporated into an ointment consisting of a white wax soft paraffin base, or as hydrogel with cellulose or polyacrylate derivatives or other viscosity modifiers, or as a dry powder or liquid spray or aerosol with butane/propane, HFA or CFC propellants, or as a drug-incorporated dressing either as a tulle dressing, with white soft paraffin or polyethylene glycols impregnated gauze dressings or with hydrogel, hydrocolloid, alginate or film dressings. The compounds could also be administered intraocularly as an eye drop with appropriate buffers, viscosity modifiers (e.g. cellulose derivatives), preservatives (e.g. benzalkonium chloride (BZK)) and agents to adjust tenicity (e.g. sodium chloride). Such formulation techniques are well-known in the art. In some instances the formulations may advantageously also contain an antibiotic. All such formulations may also contain appropriate stabilisers and preservatives.

For veterinary use, compounds can be administered as a suitably acceptable formulation in accordance with normal veterinary practice and the veterinary surgeon will determine the dosing regimen and route of administration which will be most appropriate for a particular animal.

For topical application dip, spray, powder, dust, pour-on, spot-on, emulsifiable concentrate, jetting fluid, shampoos, collar, tag or harness may be used. Such formulations are prepared in a conventional manner in accordance with standard veterinary and pharmaceutical practice. Thus capsules, boluses or tablets may be prepared by mixing the active ingredient with a suitable finely divided diluent or carrier, additionally containing a disintegrating agent and/or binder such as starch, lactose, talc, or magnesium stearate. A drench formulation may be prepared by dispersing the active ingredients in an aqueous solution together with dispersing or wetting agents and injectable formulations may be prepared in the form of a sterile solution or emulsion. Pour-on or spot-on formulations may be prepared by dissolving the active ingredients in an acceptable liquid carrier vehicle, such as butyl digol, liquid paraffin or non-volatile ester with or without addition of a volatile component such as isopropanol.

Alternatively, pour-on, spot-on or spray formulations can be prepared by encapsulation to leave a residue of active agent on the surface of the animal. These formulations will vary with regard to the weight of active compound depending on the species of host animal to be treated, the severity and type of infection and type and body weight of the host. The combinations may be administered continuously, particularly for prophylaxis by known methods. Generally for oral, parenteral and pour-on administration a dose of from about 0.001 to 10 mg per kg of animal body weight given as a single dose or in divided doses for a period of from 1 to 5 days will be satisfactory but of course there can be instances where higher or lower dosage ranges are indicated and such are within the scope of this invention.

As an alternative the combinations may be administered with the animal feedstuff and for this purpose a concentrated feed additive or premix may be prepared for mixing with the normal animal feed.

For use as an insecticide and for treating agricultural pests the compounds are applied as sprays, dusts, pour-on formulations, emulsions and the like in accordance with standard agricultural practice.

For human use the combinations are administered as a pharmaceutically acceptable formulation in accordance with normal medical practice.

The combinations of compounds of the invention may be formulated as described above as a mixture, alternatively the avermectin or milbemycin compound and the bis-aryl compound may be administered as separate doses and such treatment is still within the scope of the invention. Thus, according to another aspect of the invention, there is provided a method of combatting parasites, especially in a human or non-human animal patient which comprises administering to the patient an effective amount of a combination as described above in either single or separate doses.

The compositions, treatments, etc, of the invention may be combined with other agents, treatments, etc. useful against certain other diseases or in the reduction or suppression of other symptoms. Examples of such agents (which are provided by way of illustration and should not be construed as limiting) include other antiparasitics, eg lufenuron, imidacloprid, organophosphates, pyrethroids; antihistamines, eg chlorpheniramine, trimeprazine, diphenhydramine, doxylamine; antifungals, eg fluconazole, ketoconazole, itraconazole, griseofulvin, amphotericin B; antibacterials, eg enroflaxacin, marbofloxacin, ampicillin, amoxycillin; anti-inflammatories eg prednisolone, betamethasone, dexamethasone, carprofen, ketoprofen; dietary supplements, eg gamma-linoleic acid; and emollients. Therefore, the invention further provides a product containing a compound of the invention and one or more selected compounds from the above list as a combined preparation for simultaneous, separate or sequential use in the treatment of conditions mediated by parasites, and the related methods of treatment, etc.

The avermectin or milbemycin or derivative may be selected from those known in the art. Suitable compounds include ivermectin (22,23-dihydroavermectin B₁) described in EP 1689A and doramectin and its analogues described in EP-0214731B; the avermectin monosaccharide derivatives described in International Patent Application PCT/EP94/00095; Milbemycin D (antibiotic B41D) and its analogues described in U.S. Pat. No. 3,950,360; the nemadectins described in EP-170006A and moxidectin, and related derivatives described in EP-259779A. A number of avermectins and milbemycins have been commercialised, such as ivermectin (Ivomec™), doramectin (Dectomax™), Milbemycin D, moxidectin, selamectin (Revolution™, Stronghold™), abamectin, eprinomectin (Eprinex™) and these form a preferred group of such compounds.

Preferred bis-aryl compounds may be selected from the insecticidal and acaricidal bis-aryl compounds mentioned above. These include the aryl pyrazoles, such as the N-phenylpyrazoles described in EP-295117 including, most preferably, 5-amino-3-cyano-1-(2,6-dichloro-4-trifluorome-thylphenyl)-4-trifluoromethylsulphinylpyrazole), a compound known as fipronil. Further preferred aryl pyrazoles include those generically disclosed and specifically exemplified in the following patent applications: WO 97/07102; EP 0 846 686 A1; WO 98/04530; WO 98/24767; EP 0933 363 A1; EP 0957 094 A1 and EP 0959071 A1.

In particular, WO 97/07102 discloses bis-aryl compounds which include the following, designated herein as Formula (I):

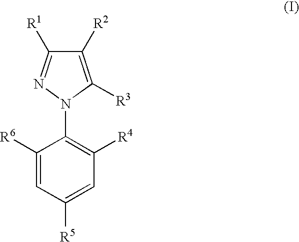

wherein

R¹ represents CN, C₁₋₆ alkoxycarbonyl, NO₂, CHO, C₁₋₆ alkanoyl, phenyl optionally substituted by one or more halogen, or C₁₋₆ alkyl optionally substituted by one or more halogen;

R² represents a group of formula II, III or IV, 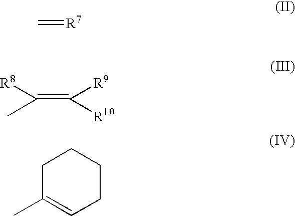
in which

R⁷ represents H, halogen, carbamoyl cyano, tri(C₁₋₆ alkyl)silyl, C₁₋₆ alkyl (optionally substituted by one or more halogen, OH or C₁₋₆ alkoxy), C₁₋₆alkoxycarbonyl, phenyl, or a 5- or 6-membered ring heterocycle which is saturated or partially or fully unsaturated and contains up to 4 hetero-atoms independently selected from up to 4 N atoms, up to 2 O atoms and up to 2 S atoms and which is attached to the alkynyl moiety by an available C, S or N atom where the valence allows; and

and R⁸, R⁹ and R¹⁰ each independently represent H, halogen, phenyl optionally substituted by one or more halogen, CN or C₁₋₆ alkyl optionally substituted by one or more halogen;

R³ represents H, C₁₋₆ alkyl halogen, NH₂, NH(C₁₋₆ alkanoyl), NH(C₁₋₆ alkoxycarbonyl), N(C₁₋₆ alkoxycarbonyl)₂, NH(C₁₋₆ alkyl), N(C₁₋₆ alkyl)₂, NHCONH(C₁₋₆ alkyl), N-pyrrolyl, NHCONH(phenyl optionally substituted by one or more halogen), N═CH(phenyl), OH, C₁₋₆ alkoxy, SH or S(O)ₙ (C₁₋₆ alkyl optionally substituted by one or more halogen) where n is 0, 1 or 2; and

R⁴, R⁵ and R⁶ each independently represent H, halogen, C₁₋₄ alkyl optionally substituted by one or more halogen, C₁₋₆ alkoxy optionally substituted by one or more halogen, S(O)ₙ (C₁₋₆ alkyl optionally substituted by one or more halogen) where n is 0, 1 or 2, or CH₃ CO, CN, CONH₂, CSNH₂, OCF₃, SCF₃ or SF₅;

or a pharmaceutically or vetennarily acceptable salt thereof.

Bis-aryls according to paragraph [0027] include compounds wherein R¹ is R^{1′} is CN, optionally substituted phenyl, optionally substituted C₁₋₆ alkyl, or C₁₋₆ alkoxycarbonyl.

Bis-aryls according to paragraph [0027] include compounds wherein R² is a group of formula II where R⁷ is H, tri(C₁₋₆ alkyl)silyl, C₁₋₆ alkyl optionally substituted by one or more halogen, OH or C₁₋₆ alkoxy, or R⁷ is C₁₋₆ alkoxycarbonyl, phenyl, a 5- or 6-membered ring heterocycle, or halogen,

or R² is a group of formula III in which either R⁸, R⁹, and R¹⁰ are each H,

or a group of formula III in which two of R⁸, R⁹ and R¹⁰ are halogen and the other is H, CN, phenyl optionally substituted by one or more halogen or C₁₋₆ alkyl optionally substituted by one or more halogen,

or a group of formula III in which R⁸, R⁹ and R¹⁰ are each independently F, Cl, Br or I,

or a group of formula III in which R⁸ is H or C₁₋₆ alkyl optionally substituted by one or more halogen, OH or C₁₋₆ alkoxy, and R⁹ and R¹⁰ are both halogen,

or a group of formula III in which R⁸ is H and one of R⁹ and R¹⁰ is halogen and the other is C₁₋₆ alkyl optionally substituted by one or more halogen, OH or C₁₋₆ alkoxy,

or a group of formula III in which R⁸ is H and one of R⁹ and R¹⁰ is H and the other is CN or C₁₋₆ alkyl optionally substituted by one or more halogen, OH or C₁₋₆ alkoxy,

or a group of formula III in which R⁸ is H and R⁹ and R¹⁰ are C₁₋₆ alkyl optionally substituted by one or more halogen, OH or C₁₋₄ alkoxy,

or a group of formula III in which R⁸ is C₁₋₆ alkyl optionally substituted by one or more halogen, OH or C₁₋₆ alkoxy and R⁹ and R¹⁰ are both H,

or a group of formula IV.

Bis-aryls according to paragraph [0027] include compounds wherein R³ is H, C₁₋₆ alkyl NH₂, NH(C₁₋₆ alkanoyl), NH(C₁₋₄ alkoxycarbonyl), N(C₁₋₆ alkoxycarbonyl)₂, N(C₁₋₆ alkyl)₂, N-pyrrolyl, halogen or S(O)ₙ (C₁₋₆ alkyl optionally substituted by one or more halogen) where n is 0, 1 or 2.

Bis-aryls according to paragraph [0027] include compounds wherein R⁴ and R⁶ are each halogen.

Bis-aryls according to paragraph [0027] include compounds wherein R⁵ is C₁₋₆ alkyl optionally substituted by one or more halogen, C₁₋₆ alkoxy optionally substituted by one or more halogen, C₁₋₆ alkylthio optionally substituted by one or more halogen, SF₅ or halogen.

Bis-aryls according to paragraph [0027] include a compound selected from:
3-cyano-1-(2,6-dichloro-4-trifluoromethylphenyl)-4-ethynylpyrazole;
3-cyano-1-(2,6-dichloro-4-trifluoromethoxyphenyl)-4-ethynylpyrazole;
3-cyano-1-(2,6-dichloro-4-trifluoromethylsulphenylphenyl)-4-ethynylpyrazole;
4-(2-bromo-1,2-dichloroethenyl)-3-cyano-1-(2,6-dichloro-4-trifluoromethoxyp henyl)-pyrazole;
3-cyano-1-(2,6-dichloro-4-trifluoromethoxyhenyl)-4-tribromoethenylpyrazole;
4-(2,2-dibromoethenyl)-3-cyano-1-(2,6-dichloro-4-trifluoromethylphenyl)-pyrazole;
3-cyano-4-(2,2-dichloroethenyl)-1-(2,6-dichloro-4-trifluoromethylphenyl)pyrazole,
3-cyano-1-(2,6-dichloro-4-trifluoromethylphenyl)-4-(2,2-difluoroethenyl)pyrazole;
3-cyano 1-(2,6-dichloro-4-trifluoromethylphenyl)-4-tribromoethenylpyrazole;
3-cyano-1-(2,6-dichloro-4-trifluoromethylphenyl)-4-trichloroethenylpyrazole;
4-(2-bromo-1,2-dichloroethenyl)-3-cyano-1-(2,6-dichloro-4-trifluoromethylphenyl)-pyrazole;
4-(2-chloro-1,2-dibromoethenyl)-3-cyano-1-(2,6-dichloro-4-trifluoromethylphenyl)-pyrazole;
3-cyano-1-(2,6-dichloro-4-trifluoromethylphenyl)-4-(1-methyl-2,2-dibromoeth enyl)pyrazole;
3-cyano 1-(2,6-dichloro-4-trifiluoromethylphenyl)-4-(1-methyl-2,2-difluoroet henyl)pyrazole;
1-(2,6-dichloro-4-trifluoromethylphenyl)-4-ethynyl-3-trifluoromethylpyrazole;
4-(2-bromo-1,2-dichloroethenyl)-1-(2,6-dichloro-4-trifluoromethylphenyl)-3-trifluoromethylpyrazole; and
1-(2,6-dichloro-4-trifluoromethylphenyl)-4-ethynyl-3-methylpyrazole, or salt thereof.

EP0846686 discloses bis-aryl compounds which include the following, designated herein as Formula (I′):

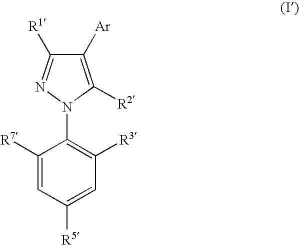

wherein:

R^{1′} is H, C₁₋₆ alkyl optionally substituted by one or more halogen atoms, halogen, CN, C₁₋₆ alkoxycarbonyl optionally substituted by one or more halogen atoms, NO₂, CHO, CONH₂, CSNH₂ of C₁₋₆ alkanoyl optionally substituted by one or more halogen atoms;

Ar is a ring “A” where “A” is a phenyl group, or “A” is a 5- or 6-membered heteroaryl group, wherein heteroaryl means a fully unsaturated heterocycle, containing 1, 2 or 3 heteroatoms, which heteroatoms are independently selected from 1 N atom, 1 or 2 O atoms and 1 or 2 S atoms, where the valence allows, or Ar is a fused bicyclic moiety “AB” where the “A” ring is as defined above and the “B” ring fused thereto in “AB” is a 5- or 6-membered saturated or partially or fully unsaturated carbocycle, or saturated or partially or fully unsaturated heterocycle where the valence allows, which heterocycle contains 1, 2, 3 or 4 hetero-atoms independently selected from 1, 2, 3 or 4 N atoms, 1 or 2 O atoms and 1 or 2 S atoms, where the valence allows, said Ar group being linked via the “A” ring to the 4-position of the pyrazole via a carbon—carbon bond,

and said Ar group being optionally substituted by one or more substituents independently selected from halogen, C₁₋₆ alkyl optionally substituted by one or more halogen atoms, C₁₋₆ alkoxy optionally substituted by one or more halogen atoms, C₁₋₆ alkoxycarbonyl optionally substituted by one or more halogen atoms, NO₂, NH₂, CN or S(O)ₘ (C₁₋₆ alkyl wherein C₁₋₆ alkyl is optionally substituted by one or more halogen atoms);
R^{2′} is H, halogen, C₁₋₆ alkyl optionally substituted by one or more halogen atoms, C₂-6 alkenyl optionally substituted by one or more halogen atoms, C₂-6 alkynyl optionally substituted by one or more halogen atoms, NH₂, NH(C₁₋₆ alkanoyl optionally substituted by one or more halogen atoms), NH(C₁₋₆ alkoxycarbonyl optionally substituted by one or more halogen atoms), N(C₁₋₆ alkoxycarbonyl optionally substituted by one or more halogen atoms)₂, NH(C₁₋₆ alkyl optionally substituted by one or more halogen atoms), N(C₁₋₆ alkyl optionally substituted by one or more halogen atoms)₂, NHCONH(C₁₋₆ alkyl optionally substituted by one or more halogen atoms), N-pyrrolyl, NHCONH(phenyl optionally substituted by one or more halogen atoms), N═CH(phenyl optionally substituted by one or more halogen atoms), OH, C₁₋₆ alkoxy optionally substituted by one or more halogen atoms, SH or S(O)ₘ (C₁₋₄ alkyl wherein C₁₋₆ alkyl is optionally substituted by one or more halogen atoms); R^{3′}, R^{4′}, R^{5′}, R^{6′} and R^{7′} are each independently H, halogen, nitro, C₁₋₆ alkoxycarbonyl optionally substituted by one or more halogen atoms, C₁₋₆ alkyl optionally substituted by one or more halogen atoms, CN, C₁₋₆ alkanoyl optionally substituted by one or more halogen atoms, CONH₂, CSNH₂, C₁₋₆ alkoxy optionally substituted by one or more halogen atoms, S(O)ₘ (C₁₋₆ alkyl wherein C₁₋₆ alkyl is optionally substituted by one or more halogen atoms) or SF₅;

m is 0, 1 or 2;

with the proviso that when

R″ is H or C₁₋₆ alkyl,

R² is H, halogen, NH₂, NH(C₁₋₆ alkanoyl optionally substituted by one or more halogen atoms), NH(C₁₋₆ alkoxycarbonyl optionally substituted by one or more halogen atoms), N(C₁₋₆ alkoxycarbonyl optionally substituted by one or more halogen atoms)₂, NH(C₁₋₆ alkyl optionally substituted by one or more halogen atoms), NHCONH(C₁₋₆ alkyl optionally substituted by one or more halogen atoms), NHCONH(phenyl optionally substituted by one or more halogen atoms),

and R^{3′}, R^{4′}, R^{5′}, R^{6′} and R^{7′} are as defined for a compound of formula (I) then Ar is not a heteroaryl group “A”;

or a pharmaceutically- or veterinarily-acceptable salt thereof.

Bis-aryls according to paragraph [0034] include compounds wherein R^{1′} is CH₃,

R^{2′} is H or NH₂,

R^{3′} is halogen,

R^{5′} is H, C₁₋₆ alkyl optionally substituted by one or more halogen atoms, C₁₋₆ alkoxy optionally substituted by one or more halogen atoms or SF₅,

R^{7′} is halogen,

and Ar is phenyl, 3,4-methylenedioxyphenyl or naphth-1-yl, each optionally substituted by one or (independently) more halogen atoms, C₁₋₆ alkyl optionally substituted by one or more halogen atoms, C₁₋₆ alkoxy optionally substituted by one or more halogen atoms, C₁₋₆ alkoxycarbonyl optionally substituted by one or more halogen atoms, NO₂, NH₂, CN or S(O)ₘ (C₁₋₆ alkyl wherein C₁₋₆ alkyl is optionally substituted by one or more halogen atoms),

and pharmaceutically or veterinarily acceptable salts thereof.

Bis-aryls according to paragraph [0034] include compounds wherein R^{3′} is Cl.

Bis-aryls according to paragraph [0034] include compounds wherein R^{5′} is H, CH₃, CF₃, OCF₃ or SF₅.

Bis-aryls according to paragraph [0034] include compounds wherein R^{7′} is Cl

Bis-aryls according to paragraph [0034] include compounds wherein Ar is 4-methylphenyl, phenyl, 2-n-butylphenyl, 3-nitrophenyl, 4-bromophenyl, 4-chlorophenyl, 4-fluorophenyl, 3,5-dichlorophenyl, 2,3-dichlorophenyl, 3-chloro-4-fluorophenyl, 3-methoxycarbonylphenyl, 3-aminophenyl, 4-methoxyphenyl, 3,4-methylenedioxyphenyl, 3,4-dimethoxyphenyl, 3-fluorophenyl, 3-chlorophenyl, 2-fluorophenyl, 2-methoxyphenyl, 2-methylphenyl, 2-trifluoromethylphenyl, 2-chlorophenyl, or naphth-1-yl.

Bis-aryls according to paragraph [0034] include compounds wherein Ar is 2-fluorophenyl, 2-methylphenyl, 2-n-butylphenyl, or 2,3-dichlorophenyl.

WO 98/24767 discloses bis-aryl compounds which include the following, designated herein as Formula (V): 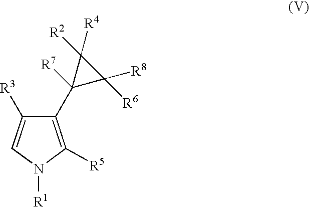
or a pharmaceutically, veterinarily or agriculturally acceptable salt thereof, or a pharmaceutically, veterinariiy or agriculturally acceptable solvate of either entity, wherein
R¹ is 2,4,6-trisubstituted phenyl wherein the 2- and 6-substituents are each independently selected from halo and the 4-substituent is selected from C₁ to C₄ alkyl optionally substituted with one or more halo, C₁ to C₄ alkoxy optionally substituted with one or more halo, S(O)nC₁ to C₄ alkyl optionally substituted with one or more halo, halo and pentafluorothio;
or 3,5-disubstituted pyridin-2-yl wherein the 3 substituent is halo and the 5-substituent is selected from C₁ to C₄ alkyl optionally substituted with one or more halo, C₁ to C₄ alkoxy optionally substituted with one or more halo, S(O)ₙC₁ to C₄ alkyl optionally substituted with one or more halo, and pentafluorothio;
R³ is C₁ to C₄ alkyl optionally substituted with hydroxy or with one or more halo; cyano, C, to C₅ alkanoyl or phenyl;
R⁵ is hydrogen, C₁ to C₄ alkyl, amino or halo;
R² and R⁴ are each independently selected from hydrogen, C₁ to C₄ alkyl, fluoro, chloro and bromo or, together with the carbon atom to which they are attached, form a C₃ to C₆ cycloalkyl group;
R⁶ and R⁸ are each independently selected from hydrogen, C₁ to C₄ alkyl, fluoro, chloro and bromo;
or, when R² and R⁴ do not form part of a cycloalkyl group, R² and R⁶, together with the carbon atoms to which they are attached, may form a C₅ to C₇ cycloalkyl group;
R⁷ is hydrogen, C, to C₄ alkyl optionally substituted with one or more halo, or C, to C₄ alkoxy; and n is 0, 1 or 2.

Bis-aryls according to paragraph [0041] include compounds wherein R¹ is 2,6-dichloro-4-trifluoromethylphenyl, 2,6-dichloro-4-pentafluorothiophenyl, 2,4,6-trichlorophenyl or 3-chloro-5-trifluoromethylpyridin-2-yl; R³ is methyl, ethyl, prop-2-yl, 1-hydroxyethyl, 2-hydroxyprop-2-yl, difluoromethyl, dichloromethyl, trifluoromethyl, cyano, formyl, acetyl or phenyl; R⁵ is hydrogen, methyl, amino or chloro; R² and R⁴ are each independently selected from hydrogen, methyl, fluoro, chloro and bromo or, together with the carbon atom to which they are attached, form a cyclopropyl, cyclobutyl or cyclopentyl group; R⁶ and R⁸ are each independently selected from hydrogen, methyl, chloro and bromo; or, when R² and R⁴ do not form part of a cycloalkyl group, R² and R⁶, together with the carbon atoms to which they are attached, may form a cyclopentane or cyclohexane group; and R⁷ is hydrogen, methyl, ethyl, trifluoromethyl, chlorodifluoromethyl, pentafluoroethyl, heptafluoropropyl or methoxy.

Bis-aryls according to pargraph [0042] include compounds wherein R¹ is 2,6-dichloro-4-trifluoromethylphenyl, 2,6-dichloro-4-pentafluorothiophenyl or 3-chloro-5 trifluoromethylpyridin-2-yl; R³ is cyano; R⁵ is hydrogen or amino; R² and R⁴ are the same and are hydrogen, chloro or bromo; R⁶ and R⁸ are hydrogen; and R⁷ is hydrogen, trifluoromethyl or chlorodifluoromethyl.

Bis aryls according to paragraph [0043] include compounds selected from:
3-cyano-4-(2,2-dibromocyclopropyl)-1-(2,6-dichloro-4-trifluoromethylphenyl)pyrazole;
(−)-3-cyano-4-(2,2-dibromocyclopropyl)-1-(2,6-dichloro-4-trifluoromethylphenyl)pyrazole;
3-cyano-1-(2,6-dichloro-4-trifluoromethylphenyl)-4-(1-trifluoromethylcyclopropyl)pyrazole;
3-cyano-4-(2,2-dibromocyclopropyl)-1-(2,6-dichloro-4-pentafluorothiophenyl)pyrazole;
3-cyano-4-(2,2-dichlorocyclopropyl)-1-(2,6-dichloro-4-trifluoromethylphenyl)pyrazole;
4-(1-chlorodifluoromethylcyclopropyl)-3-cyano-1-(2,6-dichloro-4-trifluoromethylphenyl)pyrazole;
1-[(3-chloro-5-trifluoromethyl)pyridin-2-yl]-3-cyano-4-(2,2-dibromocyclopropyl)pyrazole;
5-amino-3-cyano-4-(2,2-dibromocyclopropyl)-1-(2,6-dichloro-4-trifluoromethylphenyl)pyrazole;
5-amino-3-cyano-4-(2,2-dibromocyclopropyl)-1-(2,6-dichloro-4-pentafluorothiophenyl)pyrazole;
5-amino-3-cyano-4-(2,2-dichlorocyclopropyl)-1-(2,6-dichloro-4-pentafluorothiophenyl)pyrazole; and
5-amino-3-cyano-1-(2,6-dichloro-4-trifluoromethylphenyl)-4-(1-trifluoromethylcyclopropyl)pyrazole.

EP295117 discloses bis-aryl compounds which include the following, designated herein as Formula (VI): 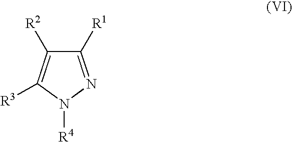
R¹ represents a cyano or nitro group, a halogen atom, or an acetyl or formyl group;
R² represents a group R⁵SO2, R⁵SO, or R⁵S in which R⁵ represents a straight- or branched-chain alkyl, alkenyl or alkynyl group containing up to 4 carbon atoms which may be unsubstituted or substituted by one or more halogen atoms which may be the same or different;
R³ represents a hydrogen atom or an amino group —NR⁶R⁷ wherein R⁶ and R⁷, which may be the same or different, each represent a hydrogen atom or a straight- or branched-chain alkyl, alkenylalkyl or alkynylalkyl group containing up to 5 carbon atoms, a formyl group, a straight- or branched-chain alkanoyl group (which contains from 2 to 5 carbon atoms and which may be optionally substituted by one or more halogen atoms) or R⁶ and R⁷ together with the nitrogen atom to which they are attached form a 5 or 6 membered cyclic imide, or represents a straight- or branched-chain alkoxycarbonyl group (which contains from 2 to 5 carbons atoms and is unsubstituted or substituted by one or more halogen atoms), or R³ represents a straight or branched-chain alkoxymethyleneamino group containing from 2 to 5 carbon atoms which may be unsubstituted or substituted on methylene by a straight or branched-chain alkyl group containing from 1 to 4 carbon atoms or represents a halogen atom; and R⁴ represents a phenyl group substituted in the 2-position by a fluorine, chlorine, bromine or iodine atom; in the 4-position by a straight- or branched-chain alkyl or alkoxy group containing from 1 to 4 carbon atoms, which may be unsubstituted or substituted by one or more halogen atoms which may be the same or different, or a chlorine or bromine atom; and optionally in the 6-position by a fluorine, chlorine, bromine or iodine atom, with the exclusion of the compound wherein R¹ represents cyano, R² represents methanesulphonyl, R³ represents amino and R⁴ represents 2,6-dichloro-4-trifluoromethylphenyl.

Bis-aryls according to paragraph [0045] include compounds wherein R² is other than a formyl group and neither R⁶ nor R⁷ represents an alkenylalkyl or alkynylalkyl group.

Bis-aryls according to paragraph [0045] include compounds wherein R² represents an alkylsulphonyl/sulphinyl/thio group which is optionally halogen substituted and contains from 1 to 4 carbon atoms, or an alkenyl- or alkynyl-sulphonyl/sulphinyl/thio group which is optionally halogen substituted and contains up to 4 carbon atoms, R³ represents the hydrogen atom, an amino or methylamino group and R¹ represents a halogen atom or a cyano or nitro group.

Bis-aryls according to paragraph [0045] include compounds wherein R¹ represents a cyano or nitro group.

Bis-aryls according to any one of paragraphs [0045]-[0048] include compounds wherein R⁴ contains a trifluoromethyl or trifluoromethoxy group and R² represents an optionally halogenated alkylsulphonyl/sulphinyl/thio group containing from 1 to 4 carbon atoms.

Bis-aryls according to paragraph [0049] include compounds wherein R² represents a trifluoromethylthio, trifluoromethylsulphinyl or trifluoromethylsulphonyl group.

Bis-aryls according to any one of paragraphs [0045]-[0048] include compounds wherein R⁴ represents a 2,4,6-trichloro-, 2,6-dichloro-4-difluoromethoxy-, 2-chloro-4-trifluoromethyl-, 2-bromo-6-chloro-4-trifluoromethyl-, 2,6-dibromo-4-trifluoromethyl- or 2-bromo-4-trifluoromethyl-phenyl group.

Bis-aryls according to any one of paragraphs [0045]-[0048] include compounds wherein R⁴ represents a 2,6-dichloro 4-trifluoromethyl- or 2,6-dichloro-4-trifluoromethoxy-phenyl group.

Bis-aryls according to paragraph [0045] include a compound selected from the group consisting of:
5-amino-3-cyano-1-(2,6-dichloro-4-trifluoromethylphenyl)-4-trifluoromethylthiopyrazole;
5-amino-3-cyano-1-(2,6-dichloro-4-trifluoromethoxyphenyl)-4-trifluoromethylthiopyrazole;
5-amino-3-cyano-1-(2,6-dichloro-4-difluoromethoxyphenyl)-4-trifluoromethylthiopyrazole;
5-amino-1-(2-chloro-4-trifluoromethylphenyl)-3-cyano-4-trifluoromethylthiopyrazole;
5-amino-3-cyano-1-(2,4,6-trichlorophenyl)-4-trifluoromethylthiopyrazole;
5-amino-3-cyano-1-(2,6-dibromo-4-trifluoromethylphenyl)-4-trifluoromethylthiopyrazole;
5-amino-1-(2-bromo-4-trifluoromethylphenyl)-3-cyano-4-trifluoromethylthiopyrazole;
5-amino-3-cyano-1-(2,6-dichloro-4-trifluoromethylphenyl)-4-difluoromethylthiopyrazole;
5-amino-3-cyano-1-(2,6-dichloro-4-trifluoromethylphenyl)-4-heptafluoropropylthiopyrazole;
5-amino-1-(2-bromo-6-chloro-4-trifluoromethylphenyl)-3-cyano-4-trifluoromethylthiopyrazole;
5-amino-3-cyano-1-(2,6-dichloro-4-trifluoromethylphenyl)-4-trichloromethylthiopyrazole;
5-amino-3-chloro-1-(2,6-dichloro-4-trifluoromethylphenyl)-4-trifluoromethylthiopyrazole;
5-amino-3-bromo-1-(2,6-dichloro-4-trifluoromethylphenyl)-4-trifluoromethylthiopyrazole;
5-amino-1-(2,6-dichloro-4-trifluoromethylphenyl)-3-fluoro-4-trifluoromethylthiopyrazole;
5-amino-4-chlorodifluoromethylthio-3-cyano1-(2,6-dichloro-4-trifluoromethylphenyl)pyrazole;
5-chloro-3-cyano-1-(2,6-dichloro-4-trifluoromethylphenyl)-4-trifluoromethylthiopyrazole;
5-amino-3-chloro-1-(2,6-dichloro-4-trifluoromethylphenyl)-4-methanesulphonylpyrazole;
3-cyano-1-(2,6-dichloro-4-trifluoromethylphenyl)-5-ethoxymethyleneamino-4-trifluoromethylthiopyrazole;
3-cyano-1-(2,6-dichloro-4-trifluoromethylphenyl)-5-ethoxyethylideneamino-4-trifluoromethylthiopyrazole;
3-cyano-1-(2,6-dichloro-4-trifluoromethylphenyl)-5-ethoxymethyleneamino-4-methanesulphonylpyrazole;
5-acetamido-3-cyano-1-(2,6-dichloro-4-trifluoromethylphenyl)-4-trifluoromethylthiopyrazole;
3-cyano-1-(2,6-dichloro-4-trifluoromethylphenyl)-5-bis(propionyl)amino-4-trifluoromethylthiopyrazole;
3-cyano-1-(2,6-dichloro-4-trifluoromethylphenyl)-5-propionamido-4-trifluoromethylthiopyrazole;
5-acetamido-3-cyano-1-(2,6-dichloro-4-trifluoromethylphenyl)-4-methanesulphonylpyrazole;
3-cyano-1-(2,6-dichloro-4-trifluoromethylphenyl)-4-trifluoromethylthio-5-trimethylacetamidopyrazole;
3-cyano-1-(2,6-dichloro-4-trifluoromethylphenyl)-5-bis(methoxycarbonyl)amino-4-trifluoromethylthiopyrazole;
3-cyano-1-(2,6-dichloro-4-trifluoromethylphenyl)-5-bis(ethoxycarbonyl)amino-4-trifluoromethylthiopyrazole;
5-chloroacetamido-3-cyano-1-(2,6-dichloro-4-trifluoromethylphenyl-4-trifluoromethylthiopyrazole;
3-cyano-1-(2,6-dichloro-4-trifluoromethylphenyl)-5-bis(ethoxycarbonyl)amino-4-methanesulphonylpyrazole;
3-cyano-1-(2,6-dichloro-4-trifluoromethylphenyl)-4-methanesulphonyl-5-trimethylacetamidopyrazole;
3-cyano-1-(2,6-dichloro-4-trifluoromethylphenyl)-5-dimethylamino-4-trifluoromethylthiopyrazole;
3-cyano-1-(2,6-dichloro-4-trifluoromethylphenyl)-5-isopropylamino-4-trifluoromethylthiopyrazole;
3-cyano-1-(2,6-dichloro-4-trifluoromethylphenyl)-5-propylamino-4-trifluoromethylthiopyrazole;
3-cyano-1-(2,6-dichloro-4-trifluoromethylphenyl)-5-dipropylamino-4-trifluoromethylthiopyrazole;
3-cyano-1-(2,6-dichloro-4-trifluoromethylphenyl)-5-bis(propargyl)amino-4-trifluoromethylthiopyrazole;
3-cyano-1-(2,6-dichloro-4-trifluoromethylphenyl)-5-methylamino-4-methanesulphonylpyrazole;
5-bromo-3-cyano-1-(2,6-dichloro-4-trifluoroethylphenyl)-4-trifluoromethanesulphonylpyrazole;
5-bromo-3-cyano-1-(2,6-dichloro-4-trifluoromethylphenyl)-4-trifluoromethylthiopyrazole;
5-bromo-3-cyano-1-(2,6-dichloro-4-trifluoromethylphenyl)-4-methanesulphonylpyrazole;
5-amino-3-bromo-1-(2,6-dichloro-4-trifluoromethylphenyl)-4-methanesulphonylpyrazole;
3-bromo-1-(2,6-dichloro-4-trifluoromethylphenyl)-4-methanesulphonylpyrazole;
3-cyano-1-(2,6-dichloro-4-trifluoromethylphenyl)-4-trifluoromethylthiopyrazole;
3-cyano-1-(2,6-dichloro-4-trifluoromethylphenyl)-4-trifluoromethanesulphonylpyrazole;
3-cyano-1-(2,6-dichloro-4-trifluoromethoxyphenyl)-4-trifluoromethylthiopyrazole;
3-cyano-1-(2,6-dichloro-4-trifluoromethylphenyl)-4-methanesulphonylpyrazole;
3-cyano-1-(2,6-dichloro-4-trifluoromethylphenyl)-5-iodo-4-trifluoromethylthiopyrazole;
3-cyano-1-(2,6-dichloro-4-trifluoromethylphenyl)-5-iodo-4-trifluoromethanesulphonylpyrazole;
5-amino-1-(2,6-dichloro-4-trifluoromethylphenyl)-3-iodo-4-methanesulphonylpyrazole;
1-(2,6-dichloro-4-trifluoromethylphenyl)-3-iodo-4-methanesulphonylpyrazole;
1-(2,6-dichloro-4-trifluoromethylphenyl)-3-iodo-4-trifluoromethylthiopyrazole;
5-amino-3-cyano-1-(2,6-dichloro-4-trifluoromethylphenyl)-4-trifluoromethanesulphonylpyrazole;
5-amino-3-cyano-1-(2,6-dichloro-4-trifluoromethylphenyl)-4-trifluoromethylsulphinylpyrazole;
5-amino-3-cyano-1-(2,6-dichloro-4-trifluoromethoxyphenyl)-4-trifluoromethanesulphonylpyrazole;
5-amino-3-cyano-1-(2,6-dichloro-4-trifluoromethoxyphenyl)-4-trifluoromethylsulphinylpyrazole;
3-cyano-1-(2,6-dichloro-4-trifluoromethylphenyl)-4-trifluoromethylsulphinylpyrazole;
5-amino-3-cyano-1-(2,6-dichloro-4-trifluoromethylphenyl)-4(1-methylprop-2-ynylsulphinyl)pyrazole;
5-amino-3-cyano-1-(2,6-dichloro-4-trifluoromethylphenyl)-4-methylsulphinylpyrazole;
5-amino-3-cyano-1-(2,6-dichloro-4-trifluoromethylphenyl)-4-isopropylsulphinylpyrazole;
5-amino-3-bromo-1-(2,6-dichloro-4-trifluoromethylphenyl)-4-trifluoromethylsulphinylpyrazole;
5-amino-4-tert-butanesulphonyl-3-cyano-1-(2,6-dichloro-4-trifluoromethylphenyl)pyrazole;
3-cyano-1-(2,6-dichloro-4-trifluoromethylphenyl)-5-propylamino-4-trifluoromethylsulphonylpyrazole;
5-acetamido-3-cyano-1-(2,6-dichloro-4-trifluoromethylphenyl)-4-trifluoromethanesulphonylpyrazole;
1-(2,6-dichloro-4-trifluoromethylphenyl)-4-methanesulphonyl-3-nitropyrazole;
5-amino-1-(2,6-dichloro-4-trifluoromethylphenyl)-4-methanesulphonyl-3-nitropyrazole;
1-(2,6-dichloro-4-trifluoromethylphenyl)-3-nitro-4-trifluoromethylsulphinylpyrazole;
5-amino-1-(2-bromo-6-chloro-4-trifluoromethylphenyl)-3-cyano-4-methanesulphonylpyrazole;
5-amino-1-(2,6-dichloro-4-trifluoromethoxyphenyl)-3-cyano-4-methanesulphonylpyrazole;
3-acetyl-5-amino-1-(2,6-dichloro-4-trifluoromethylphenyl)-4-methanesulphonylpyrazole;
5-amino-3-cyano-1-(2,6-dichloro-4-trifluoromethylphenyl)-4-methylthiopyrazole;
5-amino-3-cyano-1-(2,6-dichloro-4-trifluoromethylphenyl)-4-ethylthiopyrazole;
5-amino-3-cyano-1-(2,6-dichloro-4-trifluoromethylphenyl)-4-propylthiopyrazole;
5-amino-3-cyano-1-(2,6-dichloro-4-trifluoromethylphenyl)-4-isopropylthiopyrazole;
5-amino-3-cyano-1-(2,6-dichloro-4-trifluoromethylphenyl)-4-(2-methylpropylthio)pyrazole;
5-amino-3-cyano-1-(2,6-dichloro-4-trifluoromethylphenyl)-4(1-methylpropylthio)pyrazole;
4-allylthio-5-amino-3-cyano-1-(2,6-dichloro-4-trifluoromethylphenyl)pyrazole;
5-amino-3-cyano-1-(2,6-dichloro-4-trifluoromethylphenyl)-4-(prop-2-ynylthio)pyrazole;
5-amino-3-cyano-1-(2,6-dichloro-4-trifluoromethylphenyl)-4-(1-methylprop-2-ynylthio)pyrazole;
5-amino-3-bromo-1-(2,6-dichloro-4-trifluoromethylphenyl)-4-methylthiopyrazole;
5-amino-3-cyano-1-(2,6-dichloro-4-trifluoromethylphenyl)-4-tert-butylthiopyrazole;
5-amino-3-bromo-1-(2,6-dichloro-4-trifluoromethylphenyl)-4-methylsulphinylpyrazole;
5-amino-3-cyano-1-(2,6-dichloro-4-trifluoromethylphenyl)-4-ethanesulphonylpyrazole;
3-cyano-1-(2,6-dichloro-4-trifluoromethylphenyl)-5-methylamino-4-trifluoromethylthiopyrazole;
3-cyano-1-(2,6-dichloro-4-trifluoromethylphenyl)-5-(n-ethoxycarbonyl-n-methyl)amino-4-trifluoromethylthiopyrazole;
3-cyano-1-(2,6-dichloro-4-trifluoromethylphenyl)-5-trifluoroacetamido-4-trifluoromethylthiopyrazole;
3-cyano-1-(2,6-dichloro-4-trifluoromethylphenyl)-5-(ethoxycarbonylamino)-4-trifluoromethylthiopyrazole;
3-acetyl-1-(2,6-dichloro-4-trifluoromethylphenyl)-4-trifluoromethylthiopyrazole;
1-(2,6-dichloro-4-trifluoromethylphenyl)-3-formyl-4-trifluoromethylthiopyrazole;
5-amino-1-(2,6-dichloro-4-trifluoromethylphenyl)-3-formyl-4-trifluoromethylthiopyrazole;
3-cyano-1-(2,6-dichloro-4-trifluoromethylphenyl)-5-fluoro-4-trifluoromethanesulphonylpyrazole;
5-amino-3-cyano-4-dichlorofluoromethylthio-1-(2,6-dichloro-4-trifluoromethylphenyl)pyrazole;
5-amino-3-chloro-1-(2,6-dichloro-4-trifluoromethylphenyl)-4-trifluoromethanesulphonylpyrazole;
5-amino-3-cyano-1-(2,6-dichloro-4-trifluoromethylphenyl)-4-pentafluoroethylthiopyrazole;
3-cyano-1-(2,6-dichloro-4-trifluoromethylphenyl)-5-dimethylamino-4-trifluoromethylsulphinylpyrazole;
3-cyano-1-(2,6-dichloro-4-trifluoromethylphenyl)-5-iodo-4-trifluoromethylsulphinylpyrazole;
5-bromo-3-cyano-1-(2,6-dichloro-4-trifluoromethylphenyl)-4-trifluoromethylsulphinylpyrazole;
5-acetamido-3-cyano-1-(2,6-dichloro-4-trifluoromethylphenyl)-4-trifluoromethylsulphinylpyrazole;
3-cyano-1-(2,6-dichloro-4-trifluoromethylphenyl)-5-bis(ethoxycarbonyl)amino-4-trifluoromethanesulphonylpyrazole;
3-cyano-1-(2,6,dichloro-4-trifluoromethylphenyl)-5-ethoxycarbonylamino-4-trifluoromethanesulphonylpyrazole;
3-cyano-1-(2,6-dichloro-4-trifluoromethylphenyl)-5-ethoxymethyleneamino-4-trifluoromethanesulphonylpyrazole;
5-amino-4-(2-chloro-1,1,2-trifluoroethylthio)-3-cyano-1-(2,6-dichloro-4-trifluoromethylphenyl)pyrazole; and
3-cyano-1-(2,6-dichloro-4-trifluoromethylphenyl)-5-dimethylamino-4-trifluoromethanesulphonylpyrazole.

WO 98/04530 discloses bis-aryl compounds which include the following, designated herein as Formula (VII): 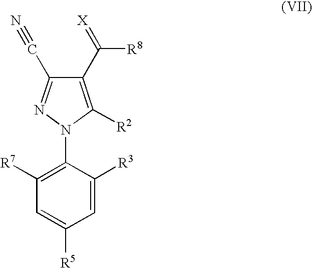
wherein
R² is NH₂, H, halogen, NH(C₁₋₆ alkyl optionally substituted with one or more halogen),
NH(allyl optionally substituted with one or more halogen), NH(benzyl optionally substituted with one or more halogen), NHSCF₃, or R² is C₁₋₆ alkyl optionally substituted with one or more halogen;
R³, R⁵ and R⁷ are each independently H, halogen, SF₅, C₁₋₆ alkyl optionally substituted with one or more halogen, C₁₋₆ alkoxy optionally substituted with one or more halogen, or S(O)ₙ(C₁₋₆ alkyl wherein C₁₋₄ alkyl is optionally substituted with one or more halogen) wherein n is 0, 1 or 2;
X is O or NOY;
Y is H or C₁₋₄ alkyl optionally substituted with one or more halogen; R is H, C₁₋₈ alkyl optionally substituted with one or more halogen, or
C₃₋₈ cycloalkyl optionally substituted by one or more halogen, or by one or more C₁₋₄ alkyl optionally substituted with one or more halogen, or by C₃₋₈ cycloalkylcarbonyl; with the proviso that the compound for formula (VII) is not 3-cyano-4-formyl-1-phenylpyrazole; or a pharmaceutically- or veterinarily-acceptable acid addition salt thereof.

Bis-aryls according to paragraph [0054] include compounds wherein R² is NH₂, H, halogen, NH(C₁₋₆ alkyl optionally substituted with one or more halogen), NH(allyl optionally substituted with one or more halogen), NH(benzyl optionally substituted with one or more halogen), NHSCF₃, or R₂ is C₁₋₆ alkyl optionally substituted with one or more halogen.

Bis-aryls according to paragraph [0054] include compounds wherein R₃ and R₇ are halogen.

Bis-aryls according to paragraph [0054] include compounds wherein R⁵ is SF₅, C₁₋₄ alkyl optionally substituted with one or more halogen or C₁₋₆ alkoxy optionally substituted with one or more halogen.

Bis-aryls according to paragraph [0054] include compounds wherein X is O or NOY where Y is H or C₁₋₆ alkyl.

Bis-aryls according to paragraph [0054] include compounds wherein R⁸ is H, C₁₋₈ alkyl optionally substituted with one or more halogen, or C₃₋₈ cycloalkyl optionally substituted by one or more C₁₋₄ alkyl.

Bis-aryls according to paragraph [0054] include compounds wherein R² is H, NHCH₃, NHCH₂Ph, NHCH₂CH═CH₂, NHCH(CH₃)₂, NHSCF₃ or NH₂.

Bis-aryls according to paragraph [0054] include compounds wherein R³ and R⁷ are Cl.

Bis-aryls according to paragraph [0054] include compounds wherein R⁵ is OCF₃, CF₃ or SF₅.

Bis-aryls according to paragraph [0054] include compounds wherein X is O, NOH or NOCH₃.

Bis-aryls according to paragraph [0054] include compounds wherein R⁸ is H, CH₃, CF₃, CH(CH₃)₂, cyclobutyl, 2,2,3,3-tetramethylcyclopropyl, C₂F₅, C₃F₇, C₂H₅, CF₂Cl, CCl₂F or cyclopropyl.

Bis-aryls according to paragraph [0054] include compounds wherein X is O.

Bis-aryls according to paragraph [0054] include a compound selected from:
4-acetyl-5-amino-3-cyano-1-(2,6-dichloro-4-trifluoromethylphenyl)pyrazole;
4-acetyl-3-cyano-1-(2,6-dichloro-4-trifluoromethylphenyl)pyrazole;
5-amino-3-cyano-1-(2,6-dichloro-4-trifluoromethylphenyl)-4-(1-oximinoethyl)pyrazole;
5-amino-3-cyano-1-(2,6-dichloro-4-trifluoromethylphenyl)-4-(1-methoximinoethyl)pyrazole;
S-amino-3-cyano-1-(2,6-dichloro-4-trifluoromethylphenyl)-4-methoximinomethylpyrazole;
5-amino-3-cyano-1-(2,6-dichloro-4-trifluoromethylphenyl)-4-trifluoroacetylpyrazole;
3-cyano-1-(2,6-dichloro-4-trifluoromethylphenyl)-4-oximinomethylpyrazole;
3-cyano-1-(2,6-dichloro-4-trifluoromethylphenyl)-4-(2-methylpropanoyl)pyrazole;
3-cyano-4-cyclopropylcarbonyl-1-(2,6-dichloro-4-trifluoromethylphenyl)pyrazole;
4-acetyl-5-amino-3-cyano-1-(2,6-dichloro-4-sulphurpentafluorophenyl)pyrazole;
4-acetyl-3-cyano-1-(2,6-dichloro-4-sulphurpentafluorophenyl)pyrazole;
4-acetyl-5-amino-3-cyano-1-(2,6-dichloro-4-trifluoromethoxyphenyl)pyrazole;
4-acetyl-3-cyano-1-(2,6-dichloro-4-trifluoromethoxyphenyl)pyrazole;
3-cyano-1-(2,6-dichloro-4-trifluoromethylphenyl)-5-methylamino-4-trifluoroacetylpyrazole;
3-cyano-1-(2,6-dichloro-4-trifluoromethylphenyl)-5-(prop-2-enylamino)-4trifluoroacetylpyrazole;
3-cyano-1-(2-(2,6-dichloro-4-trifluoromethylphenyl)-5-phenylmethylamino-4trifluoroacetylpyrazole; and
3-cyano-1-(2,6-dichloro-4-trifluoromethylphenyl)-5-(prop-2-ylamino)-4-trifluoroacetylpyrazole.

EP 0 933 363 A1 discloses bis-aryl compounds which include the following, designated herein as Formula (VIII): 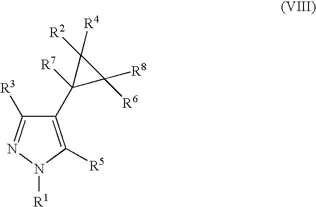
or a pharmaceutically, veterinarily or agriculturally acceptable salt thereof, or a pharmaceutically, veterinarily or agriculturally acceptable solvate of either entity, wherein R¹ is 2,4,6-trisubstituted phenyl wherein the 2- and 6-substituents are each independently selected from halo and the 4-substituent is selected from C₁ to C₄ alkyl optionally substituted with one or more halo, halo and pentafluorothio; or 3,5-disubstituted pyridin-2-yl wherein the 3-substituent is halo and the 5-substituent is selected from C₁ to C₄ alkyl optionally substituted with one or more halo, halo and pentafluorothio;
R³ is hydrogen; C₂ to C₅ alkyl substituted with one or more halo and with hydroxy; C₂ to C₅ alkanoyl substituted with one or more halo; C₂ to C₆ alkenyl optionally substituted with one or more halo; halo; amino or CONH₂;
R⁵ is hydrogen, amino or halo;
R² and R⁴ are each independently selected from hydrogen, fluoro, chloro and bromo;
R⁶ and R⁸ are hydrogen; and R⁷ is hydrogen or C₁ to C₄ alkyl optionally substituted with one or more halo.

Bis-aryls according to paragraph [0067] includes compounds wherein R¹ is 2,6-dichloro-4-trifluoromethylphenyl, 2,6-dichloro-4-pentafluorothiophenyl, 2,4,6-trichlorophenyl or 3-chloro-5-trifluoromethylpyridin-2-yl; R³ is hydrogen; hydroxytrihaloethyl; trihaloacetyl; C₂ to C₃ alkenyl optionally substituted with one or more halo; halo; amino or CONH₂; R⁵ is hydrogen or amino; R² and R⁴ are each independently selected from hydrogen, chloro and bromo; and R⁷ is hydrogen or trifluoromethyl.

Bis-aryls according to paragraph [0068] includes compounds wherein R¹ is 2,6-dichloro-4-trifluoromethylphenyl or 3-chloro-5-trifluoromethylpyridin-2-yl; R³ is hydrogen; 1-hydroxy-2,2,2-trifluoroethyl; trifluoroacetyl; ethenyl; 2,2-difluoroethenyl; 2,2-dibromoethenyl; propen-2-yl; chloro; bromo; iodo; amino or CONH₂; and R² and R⁴ are bromo.

Bis-aryls according to paragraph [0069] includes compounds wherein R³ is hydrogen; ethenyl; 2,2-difluoroethenyl; chloro; bromo or CONH₂.

EP-500209A discloses bis-aryl compounds which include the following, designated herein as Formula (IX): 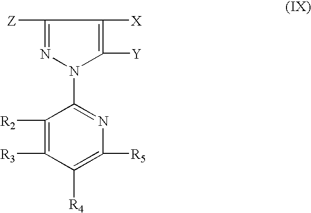
wherein:
X is a halogen, nitro or unsubstituted or halo-substituted alkylsulfenyl, alkylsulfinyl or alkylsulfonyl, wherein the alkyl moiety is a linear or branched chain, containing one to four carbon atoms, and where substituted, is substituted by one of more halogen atoms, which are the same or different, up to full substitution of the alkyl moiety;
Y is hydrogen, halogen, cyano, alkylsulfenyl, alkylsulfinyl, alkylsulfonyl, alkoxy, amino, alkylamino, dialkylamino, trialkylammonium salt, cyanoalkylamino, alkoxyalkylamino, alkoxycarbonylamino, alkylcarbonylamino, haloalkylcarbonylamino, alkylaminocarbonylamino, dialkylaminocarbonylamino or alkoxyalkylideneimino, wherein the alkyl and alkoxy moieties are linear or branched and contain one to four carbon atoms, and the haloalkyl moieties are substituted by one or more halogen atoms, which are the same or different, up to full substitution of the alkyl moiety;
Z is cyano or halogen; and
R₂, R₃, R₄ and R₅ are each individually hydrogen, halogen, alkyl, haloalkyl, alkoxy, haloalkoxy, cyano or nitro, wherein the alkyl and alkoxy moieties are linear or branched and contain one to four carbon atoms, and the halo-substituted moieties are substituted by one or more halogen atoms, which are the same or different, up to full substitution of the alkyl and alkoxy moieties; at least one of R₂ to R₅ being other then hydrogen; or an acid addition salt thereof.

Bis-aryls according to paragraph [0071] include compounds of formula (IX-A), 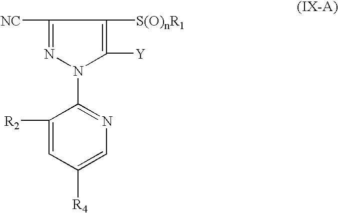
wherein Y is as defined in paragraph [0071],
R¹ is linear or branched alkyl of one of four carbon atoms, which is substituted by one or more halogen atoms, which are the same or different, up to full substitution of the alkyl group;
n is 0, 1 or 2; and
R₂ and R₄ are as defined in paragraph [0071], at least one of R₂ and R⁴ being other than hydrogen; or
an acid addition salt thereof.

Bis-aryls according to paragraph [0072] include compounds wherein: Y is amino, alkylamino, alkylsulfenyl, alkylsulfinyl, alkylsulfonyl, alkoxyalkylamino, alkylcarbonylamino, haloalkylcarbonylamino or alkoxyalkylideneimino, wherein the alkyl and alkoxy moieties are linear or branched and contain one to four carbon atoms, and the haloalkyl moieties are substituted by one or more halogen atoms, which are the same or different, up to full substitution of the alkyl moiety;

R₂ is hydrogen or halogen; and

R₄ is hydrogen, halogen, haloalkyl or haloalkoxy, wherein the haloalkyl or haloalkoxy moiety is linear or branched and contains one to four carbon atoms, and is substituted by one or more halogen atoms, which are the same or different, up to full substitution of the alkyl and alkoxy moieties; at least one of R₂ and R₄ being other than hydrogen.

Bis-aryls according to paragraph [0073] include compounds wherein:

Y is amino, alkylamino, alkoxymethylamino or alkoxyalkylideneimino wherein the alkyl and alkoxy moieties contain one or two carbon atoms;

R¹ is trifluoromethyl, dichlorofluoromethyl or chlorodifluoromethyl;

R₂ is bromine, chlorine or fluorine; and

R₄ is bromine, chlorine, fluorine, trifluoromethyl or trifluoromethoxy.

Bis-aryls according to paragraph [0074] include:
1-[2-(3-chloro-5-trifluoromethyl)pyridyl]-5-amino-3-cyano-4-chlorodifluoromethylsulfenylpyrazole;
1-[2-(3-chloro-5-trifluoromethyl)pyridyl]-5-amino-3-cyano-4-chlorodifluoromethylsulfinylpyrazole;
1-[2-(3-chloro-5-trifluoromethyl)pyridyl]-5-amino-3-cyano-4-chlorodifluoromethylsulfonylpyrazole;
1-[2-(3-chloro-5-trifluoromethyl)pyridyl]-5-amino-3-cyano-4-dichlorofluoromethylsulfenylpyrazole;
1-[2-(3-chloro-5-trifluoromethyl)pyridyl]-5-ethoxymethylamino-3-cyano-4-dichlorofluoromethylsulfenylpyrazole;
1-[2-(3-chloro-5-trifluoromethyl)pyridyl]-5-amino-3-cyano-4-trifluoromethylsulfenylpyrazole;
1-[2-(3-chloro-5-trifluoromethyl)pyridyl]-5-amino-3-cyano-4-trifluoromethylsulfinylpyrazole;
1-[2-(3-chloro-5-trifluoromethyl)pyridyl]-5-amino-3-cyano-4-trifluoromethylsulfonylpyrazole;
1-[2-(3-chloro-5-trifluoromethyl)pyridyl]-5-amino-3-cyano-4-difluoromethylsulfinylpyrazole;
1-[2-(3-chloro-5-trifluoromethyl)pyridyl]-5-amino-3-cyano-4-dichlorofluoromethylsulfonylpyrazole;
1-[2-(3-chloro-5-trifluoromethyl)pyridyl]-5-amino-3-cyano-4-methylsulfenylpyrazole;
1-[2-(3-chloro-5-trifluoromethyl)pyridyl]-5-amino-3-cyano-4-methylsulfinylpyrazole; or
1-[2-(3,5-dichloro)pyridyl]-5-amino-3-cyano-4-trifluoromethylsulfenylpyrazole; or

an acid addition salt thereof.

Bis-aryls according to paragraph [0075] include:
1-[2-(3-chloro-5-trifluoromethyl)pyridyl]-5-amino-3-cyano-4-chlorodifluoromethylsulfinylpyrazole;
1-[2-(3-chloro-5-trifluoromethyl)pyridyl]-5-amino-3-cyano-4-chlorodifluoromethylsulfonylpyrazole;
1-[2-(3-chloro-5-trifluoromethyl)pyridyl]-5-amino-3-cyano-4-trifluoromethylsulfinylpyrazole;
1-[2-(3-chloro-5-trifluoromethyl)pyridyl]-5-amino-3-cyano-4-trifluoromethylsulfonylpyrazole;
1-[2-(3-chloro-5-trifluoromethyl)pyridyl]-5-amino-3-cyano-4-difluoromethylsulfinylpyrazole; or
1-[2-(3-chloro-5-trifluoromethyl)pyridyl]-5-amino-3-cyano-4-methylsulfinylpyrazole or;

an acid addition salt thereof.

EP-511845A discloses bis-aryl compounds which include the following, designated herein as Formula (X): 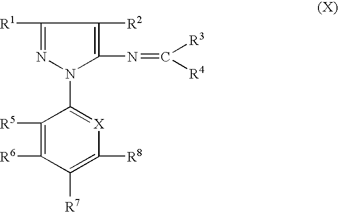
wherein:
R¹ is cyano, nitro, halogen, formyl, alkylcarbonyl or cycloalkylcarbonyl; and wherein the alkyl moieties are linear or branched chains of 14 carbon atoms and the cycloalkyl moiety contains 3 to 7 carbon atoms;
R² is: halogen; alkyl; haloalkyl; alkoxy; haloalkoxy; nitro; thiocyanato; unsubstituted or mono- or dialkyl substituted sulfamoyl; unsubstituted or mono- or dialkyl substituted aminocarbonyl; alkoxycarbonyl; or unsubstituted or substituted R⁹S(O)n, in which n is 0, 1 or 2 and R⁹ is alkyl, haloalkyl, cycloalkyl, halocycloalkyl, cycloalkylalkyl or halocycloalkylalkyl; and wherein the alkyl moieties are linear or branched chains of 14 carbon atoms, the cycloalkyl moiety contains 3 to 7 carbon atoms and the halo substitution consists of one or more halogen atoms, which are the same or different, up to full substitution of the alkyl and cycloalkyl moieties;
R³ is hydrogen, C₁₋₄ alkyl, C₁₋₄ alkoxy, C₁₋₄ alkylthio or di-C₁₋₄ alkylamino; and wherein the alkyl moieties are linear or branched chains;
   R⁴ is unsubstituted or substituted phenyl or unsubstituted or substituted heteroaryl having a five or six membered monocyclic ring containing one or more of the same or different oxygen, sulfur or nitrogen hetero atoms; and wherein the phenyl or heteroaryl substitution is one or more or combinations of: hydroxy or inorganic or organic salt thereof; sulfhydryl or inorganic or organic salt thereof; halogen; cyano; nitro; alkyl; haloalkyl; alkoxy; —O-alkyl-O—; O-haloalkyl-O—; haloalkoxy; alkanoyloxy; phenolic; trialkylsilyloxy; phenyl; alkyl-S(O)n or haloalkyl-S(O)n, in wich n is 0, 1 or 2; NR¹⁰R¹¹ in wich R¹⁰ and R¹¹ are individually hydrogen, alkyl, alkanoyl or haloalkanoyl; COR¹² in which R¹² is NR¹⁰R¹¹, alkoxy, alkylthio, hydroxy or inorganic or organic salt thereof, hydrogen, alkyl or haloalkyl; or SO₂R¹³ in which R¹³ is NR¹⁰R¹¹, alkoxy, alkylthio, or hydroxy or inorganic or organic salt thereof; and wherein the alkyl and alkoxy moieties are linear or branched chains of 1-4 carbon atoms and the halo substitution consists of one or more halogen atoms, which are the same or different, up to full substitution of the alkyl and alkoxy moieties;
   R⁵ is hydrogen, halogen or linear or branched chain C₁₋₄ alkyl;
   R⁶ and R⁸ are each individually hydrogen or fluorine;
   R⁷ is halogen, alkyl, haloalkyl, alkoxy, haloalkoxy, cyano, nitro, alkylcarbonyl, haloalkylcarbonyl, alkyl-S(O)n or haloalkyl-S(O)n in which n is 0, 1 or 2; and wherein the alkyl and alkoxy moieties are linear or branched chains of 14 carbon atoms and the halo substitution consists of one or more halogen atoms, which are the same or different, up to full substitution of the alkyl and alkoxy moieties; and
   X is a nitrogen atom (N) or C—R¹⁴ in which R¹⁴ is hydrogen, halogen, cyano, nitro, C₁₋₄ alkyl, C₁₋₄ alkylthio or C₁₋₄ alkoxy; and the alkyl moieties are linear or branched chains.

Bis-aryls according to paragraph [0077] include compounds wherein:

R¹ is cyano, nitro or halogen;

R² is unsubstituted or substituted R⁹S(O)n, in which n is 0, 1 or 2 and R⁹ is alkyl or haloalkyl as defined;

R³ is hydrogen;

R⁴ is unsubstituted or substituted phenyl or unsubstituted or substituted heteroaryl which is pyridyl, pyridyl N-oxide, thienyl, furanyl, pyrrolyl, imidazolyl or triazolyl;

R⁵ is hydrogen, halogen or alkyl;

R⁶ and R⁸ are each individually hydrogen or fluorine;

R⁷ is halogen, alkyl, haloalkyl or haloalkoxy; and

X is a nitrogen atom (N) or C—R¹⁴ in which R¹⁴ is hyrogen, halogen, cyano, alkyl, alkylthio or alkoxy.

Bis-aryls according to paragraph [0078] include compounds of formula (X-A): 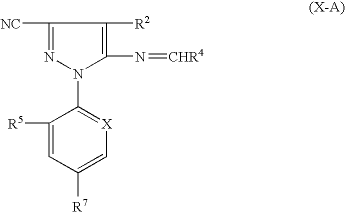
wherein:
R² is the group R⁹S(O)n, in which n is 0, 1 or 2 and R⁹ is alkyl or haloalkyl in which halo is F, Cl or Br or combinations thereof;
R⁴ is unsubstituted or substituted phenyl, in which the substituents are one or more hydroxy, halogen, alkoxy, alkylthio, cyano or alkyl or combinations thereof; or R⁴ is 4-pyridyl or 4-pyridyl N-oxide, optionally substituted as described for phenyl;
R⁵ is hydrogen, alkyl or halogen;
R⁷ is halogen, alkyl, haloalkyl or haloalkoxy, in which halo is F, Cl or Br or combinations thereof; and
X is a nitrogen atom or CR¹⁴ in which R¹⁴ is hydrogen, halogen, cyano, alkyl, alkylthio or alkoxy.

Bis-aryls according to paragraph [0079] include compounds wherein R⁴ is substituted phenyl as defined.

Bis-aryls according to paragraph [0080] include compounds wherein:

R² is the group R⁹S(O)n, in which n=0, 1 or 2 and R⁹ is alkyl, trihalomethyl or dihalomethyl;

R⁴ is substituted phenyl, in which the substituents are one or more hydroxy, F, Cl, Br, methoxy, ethoxy, methylthio, cyano, methyl or ethyl or combinations thereof;

R⁵ is hydrogen, methyl, F, Cl or Br;

R⁷ is F, Cl, Br, methyl, trihalomethyl or trihalomethoxy; and

X is a nitrogen atom or CR¹⁴ in which R¹⁴ is hydrogen, F, Cl Br, cyano, methyl, ethyl, methylthio, ethylthio, methoxy or ethoxy.

Bis-aryls according to paragraph [0081] include compounds wherein:

R² is the group R⁹S(O)n, in which n is 0, 1 or 2 and R⁹ is CH₃, CF₃ CCl₃, CF₂Cl, CFCl₂, CF₂Br, CHF₂, CHClF or CHCl₂;

R⁴ is substituted phenyl as defined;

R⁵ is as defined;

R⁷ is F, Cl, Br, CF3 or OCF3; and

X is as defined.

Bis-aryls according to any one of paragraphs [0077]-[0082] include compounds wherein R⁴ is substituted phenyl, which is at least substituted by 3-hydroxy or 4-hydroxy and the other phenyl substituents are as defined.

Bis-aryls according to paragraph [0083] include compounds wherein R⁴ is:
4-hydroxy-3-methoxyphenyl;
4-hydroxyphenyl;
3-hydroxy-4-methoxyphenyl;
3,5-dimethyl-4-hydroxyphenyl;
3,5-dimethoxy-4-hydroxyphenyl;
2,4-dihydroxyphenyl;
4-hydroxy-3-methylphenyl;
3-ethoxy-4-hydroxyphenyl;
3,4-dihydroxyphenyl;
3-chloro-4-hydroxyphenyl;
2-chloro-4-hydroxyphenyl;
5-bromo-4-hydroxy-3-methoxyphenyl;
3-hydroxyphenyl;
5-chloro-4-hydroxy-3-methoxyphenyl;
2,4,5-hydroxyphenyl;
5-bromo-3,4-dihydroxyphenyl; or
4,5-dihydroxy-3-methoxyphenyl.

Bis-aryls according to paragraph [0084] include compounds wherein the 1-aryl group 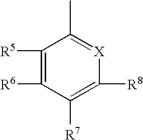
is:
2,6-dichloro-4-trifluoromethylphenyl;
2,6-dichloro-4-trifluoromethoxyphenyl;
2-chloro-4-trifluoromethoxyphenyl;
2-chloro-4-trifluoromethylphenyl;
2,4,6-trichlorophenyl;
4-bromo-2,6-dichlorophenyl;
2-chloro-6-methyl-4-trifluoromethylphenyl;
3-chloro-5-trifluoromethyl-2-pyridyl;
2-chloro-6-fluoro-4-trifluoromethylphenyl; or
2-chloro-6-cyano-4-trifluoromethylphenyl.

Bis-aryls according to paragraph [0085] include:
1-(2,6-dichloro-4-trifluoromethylphenyl)-3-cyano-4-trifluoromethylsulfenyl-5-[(4-hydroxy-3-methoxyphenyl)methylideneimino]pyrazole;
1-(2,6-dichloro-4-trifluoromethylphenyl)-3-cyano-4-trifluoromethylsulfonyl-5-[(4-hydroxy-3-methoxyphenyl)methylideneimino]pyrazole;
1-(2,6-dichloro-4-trifluoromethylphenyl)-3-cyano-4-trifluoromethylsulfenyl-5-[(4-hydroxyphenyl)methylideneimino]pyrazole;
1-(2,6-dichloro-4-trifluoromethoxyphenyl)-3-cyano-4-dichlorofluoromethylsulfenyl-5-[(4-hydroxy-3-methoxyphenyl)methylideneimino]pyrazole;
1-(2,6-dichloro-4-trifluoromethylphenyl)-3-cyano-4-dichlorofluoromethylsulfenyl-5-[(4-hydroxy-3-methoxyphenyl)methylideneimino]pyrazole;
1-(2,6-dichloro-4-trifluoromethylphenyl)-3-cyano-4-trifluoromethylsulfinyl-5-[(4-hydroxy-3-methoxyphenyl)methylideneimino]pyrazole;
1-(2,6-dichloro-4-trifluoromethylphenyl)-3-cyano-4-trifluoromethylsulfenyl-5-[(3,5-dimethoxy-4-hydroxyphenyl)methylideneimino]pyrazole;
1-(2,6-dichloro-4-trifluoromethylphenyl)-3-cyano-4-trifluoromethylsulfenyl-5-[(3-hydroxy-4-methoxyphenyl)methylideneimino]pyrazole;
1-(2,6-dichloro-4-trifluoromethylphenyl)-3-cyano-4-trifluoromethylsulfenyl-5-[(2,4-dihydroxyphenyl)methylideneimino]pyrazole;
1-(2,6-dichloro-4-trifluoromethylphenyl)-3-cyano-4-trifluoromethylsulfenyl-5-[(4-hydroxy-3-methylphenyl)methylideneimino]pyrazole;
1-(2,6-dichloro-4-trifluoromethylphenyl)-3-cyano-4-trifluoromethylsulfinyl-5-[(4-hydroxy-3-methylphenyl)methylideneimino]pyrazole;
1-(2,6-dichloro-4-trifluoromethylphenyl)-3-cyano-4-trifluoromethylsulfenyl-5-[(3,5-dimethyl-4-hydroxyphenyl)methylideneimino]pyrazole;
1-(2,6-dichloro-4-trifluoromethylphenyl)-3-cyano-4-trifluoromethylsulfinyl-5-[(3,5-dimethoxy-4-hydroxyphenyl)methylideneimino] pyrazole;
1-(2,6-dichloro-4-trifluoromethylphenyl)-3-cyano-4-trifluoromethylsulfenyl-5-[(3-ethoxy-4-hydroxyphenyl)methylideneimino] pyrazole;
1-(2,6-dichloro-4-trifluoromethylphenyl)-3-cyano-4-trifluoromethylsulfenyl-5-[(2-chloro-4-hydroxyphenyl)methylideneimino] pyrazole;
1-(2,6-dichloro-4-trifluoromethylphenyl)-3-cyano-4-trifluoromethylsulfenyl-5-[(3-chloro-4-hydroxyphenyl)methylideneimino]pyrazole; or
1-(2,6-dichloro-4-trifluoromethoxyphenyl)-3-cyano-4-dichlorofluoromethylsulfenyl-5-[(4-hydroxyphenyl)methylideneimino]pyrazole.

When used as pesticidal agent in a host animal the compositions of the invention may be applied to the human or animal host patient at a typical amount of 0.001-10 mgs/kg of body weight of avermectin or milbemycin compound. The weight ratio of avermectin or milbemycin compound to the aryl pyrazole will generally be in the range of 1 to 100, preferably 1 to 10. The synergistic effects of certain combinations allow a reduced dosage of both compounds to be employed to obtain a given antiparasitic effect, reducing risks of unwanted side effects, toxicity and development of resistance to the compounds concerned and longer duration of action.

The combination therapy of the invention is effective in treating a variety of conditions caused by endoparasites including, in particular, helminthiasis which is most frequently caused by a group of parasitic worms described as nematodes and which can cause severe economic losses in swine, sheep, horses and cattle as well as affecting domestic animals and poultry. The most common genera of nematodes infecting the animals referred to above are Haemonchus, Trichostrongylus, Ostertagia, Teladorsagia, Nematodirus, Cooperia, Ascaris, Bunostomum, Oesophagostomum, Chabertia, Strongylus, Trichonema and Dictyocaulus. The combinations are also effective against other nematodes which affect various species of animals including, for example:—Dirofilaria in dogs and various parasites which can infest livestock, companion animals such as cats and dogs and also humans including gastrointestinal parasites such as Ancylostoma, Uncinaria, Necator, Ascaris, Strongyloides, Trichinella, Capillaria, Toxocara, Toxascaris, Trichuris, Enterobius and parasites which are found in the blood or other tissues and organs such as filarial worms and the extra intestinal stages of Strongyloides, Toxocara and Trichinella. They are especially useful in treating heartworm in companion animals.

The combinations are also of value in treating ectoparasite infections including particular arthropod ectoparasites of host humans, animals and birds such as ticks, mites, lice, fleas, blowfly, biting insects and migrating dipterous larvae which can affect cattle and horses.

The combinations are also insecticides active against household pests such as the cockroach, clothes moth, carpet beetle and the housefly as well as being useful against arthropod pests of stored grain and of agricultural plants such as spider mites, aphids, caterpillars and against migratory orthopterans such as locusts.

The efficacy of compositions according to an embodiment of the invention against heartworm (Dirofilaria immitis) is demonstrated by the following Example.

### EXAMPLE

Infective L3 larvae of Dirofilaria immitis were recovered from previously infected Aedes egypti and cultured in vitro to the L4 stage by techniques described by D. Abraham et al (J. Parasit. 73(2) 1987, pp 377-383).

Test Procedure

Testing of compounds took place when >95% of the larvae had moulted to the L4 stage. The assay system consisted of a 96 well microtitre plate in which 79 mcl of assay media, 1 mcl test compound and 20 mcl assay media containing 15 to 20 L4 D. immitis were dispensed. Each test compound was dissolved in dimethylsulphoxide (DMSO) and dilutions were made using this solvent. The test microtitre plates were kept at 37 degrees centigrade under an atmosphere comprising 5% CO₂ in air for 72 h.

Assay Procedure

The effects of each compound at each concentration and combination was assessed by comparing the motility of L4 larvae in control and treated wells. Microscopic observations were made at 2, 4, 24, 48 and 72 hours on each well. Observations were scored according to the levels of larval motility, ranging from 0 (all dead) to 5 (all normal motility).

The above procedure was carried out using solutions containing the compounds shown in Table 1 below at the stated concentrations. Compound (1) is 5-oximino 22,23-dihydro-25-cyclohexylayermectin B1 monosaccharide (selamectin), disclosed as Example 5 in WO 94/15944.

TABLE 1
In vitro L4 Heartworm (D. immitis)
Duplicate Observations (hours post treatment)
Compound/s mcg/ml 2 h 4 h 24 h 48 h 72 h

| Compound/s | mcg/ml | 2 h | 4 h | 24 h | 48 h | 72 h |
|---|---|---|---|---|---|---|
| CONTROL | — | 5/5 | 5/5 | 5/5 | 5/5 | 5/5 |
| (1) | 10 | 2/2 | 2/0 | 3/3 | 4/4 | 3/4 |
| (1) | 1 | 5/5 | 5/5 | 5/5 | 5/5 | 5/5 |
| Fipronil | 10 | 5/5 | 5/5 | 5/5 | 5/5 | 5/5 |
| (1) + | 10 | 4/2 | 1/0 | 0/0 | 0/0 | 0/0 |
| Fipronil | 10 | | | | | |
| (1) + | 10 | 4/4 | 1/3 | 1/0 | 2/3 | 0/3 |
| Fibronil | 1 | | | | | |

Key - Observation Score:
0 = No motility, larvae dead
1 = Motility in at least 1 larva, all others dead
2 = >80% of larvae dead, others sluggish
3 = <80% of larvae dead, others sluggish
4 = All larvae (or most) motile but sluggish
5 = Normal in appearance and motility.

It can be seen from the results given in Table 1 that, whereas compound (1) alone had only a modest effect in causing mortality or paralysis and fipronil alone had no observable effect at all at concentrations of 10 mcg/ml, the combination of these compounds was very effective.

All publications mentioned above are herein incorporated by reference in their entirety.

Reference to treatment herein includes preventative, palliative and curative treatments.

What is claimed:

1. A composition comprising a combination of a synergistic amount of fipronil and selamectin.

2. The composition according to claim 1, wherein the composition further comprises a suitable pharmaceutical or veterinary carrier.

3. A composition comprising a combination of a synergistic amount of a bis-aryl compound of formula (I) or (I′) and selamectin, wherein the compound of formula (I) is represented by: 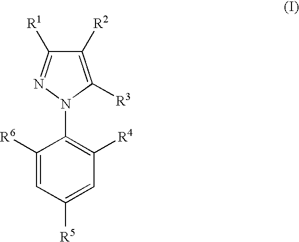
wherein
R¹ represents CN, C₁₋₆ alkoxycarbonyl, NO₂, CHO, C₁₋₆ alkanoyl, phenyl optionally substituted by one or more halogen, or C₁₋₆ alkyl optionally substituted by one or more halogen;
R² represents a group of formula II, III or IV, 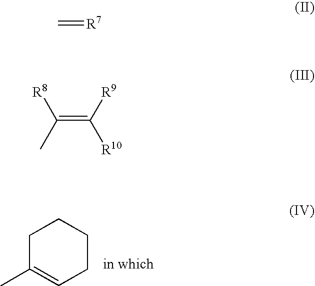
R⁷ represents H, halogen, carbamoyl cyano, tri(C₁₋₆ alkyl)silyl, C₁₋₆ alkyl (optionally substituted by one or more halogen, OH or C₁₋₆ alkoxy), C₁₋₆ alkoxycarbonyl, phenyl, or a 5- or 6-membered ring heterocycle which is saturated or partially or fully unsaturated and contains up to 4 hetero-atoms independently selected from up to 4 N atoms, up to 2 O atoms and up to 2 S atoms and which is attached to the alkynyl moiety by an available C, S or N atom where the valence allows; and
and R⁸, R⁹ and R¹⁰ each independently represent H, halogen, phenyl optionally substituted by one or more halogen, CN or C₁₋₆ alkyl optionally substituted by one or more halogen;
R³ represents H, C₁₋₆ alkyl halogen, NH₂, NH(C₁₋₆ alkanoyl), NH(C₁₋₆ alkoxycarbonyl), N(C₁₋₆ alkoxycarbonyl)₂, NH(C₁₋₆ alkyl), N(C₁₋₆ alkyl)₂, NHCONH(C₁₋₆ alkyl), N-pyrrolyl, NHCONH(phenyl optionally substituted by one or more halogen), N═CH(phenyl), OH, C₁₋₆ alkoxy, SH or S(O)ₙ (C₁₋₆ alkyl optionally substituted by one or more halogen) where n is 0, 1 or 2; and
R⁴, R⁵ and R⁶ each independently represent H, halogen, C₁₋₄ alkyl optionally substituted by one or more halogen, C₁₋₆ alkoxy optionally substituted by one or more halogen, S(O)ₙ (C₁₋₆ alkyl optionally substituted by one or more halogen) where n is 0, 1 or 2, or CH₃ CO, CN, CONH₂, CSNH₂, OCF₃, SCF₃ or SF₅;
or a pharmaceutically or vetennarily acceptable salt thereof; and wherein the compound of formula (I′) is represented by: 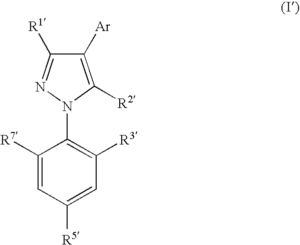
wherein:
R^{1′} is H, C₁₋₆ alkyl optionally substituted by one or more halogen atoms, halogen, CN, C₁₋₆ alkoxycarbonyl optionally substituted by one or more halogen atoms, NO₂, CHO, CONH₂, CSNH₂ of C₁₋₆ alkanoyl optionally substituted by one or more halogen atoms;
Ar is a ring “A” where “A” is a phenyl group, or “A” is a 5- or 6-membered heteroaryl group, wherein heteroaryl means a fully unsaturated heterocycle, containing 1, 2 or 3 heteroatoms, which heteroatoms are independently selected from 1 N atom, 1 or 2 O atoms and 1 or 2 S atoms, where the valence allows,
or Ar is a fused bicyclic moiety “AB” where the “A” ring is as defined above and the “B” ring fused thereto in “AB” is a 5- or 6-membered saturated or partially or fully unsaturated carbocycle, or saturated or partially or fully unsaturated heterocycle where the valence allows, which heterocycle contains 1, 2, 3 or 4 hetero-atoms independently selected from 1, 2, 3 or 4 N atoms, 1 or 2 O atoms and 1 or 2 S atoms, where the valence allows, said Ar group being linked via the “A” ring to the 4-position of the pyrazole via a carbon—carbon bond,
and said Ar group being optionally substituted by one or more substituents independently selected from halogen, C₁₋₆ alkyl optionally substituted by one or more halogen atoms, C₁₋₆ alkoxy optionally substituted by one or more halogen atoms, C₁₋₆ alkoxycarbonyl optionally substituted by one or more halogen atoms, NO₂, NH₂, CN or S(O)ₘ (C₁₋₆ alkyl wherein C₁₋₆ alkyl is optionally substituted by one or more halogen atoms);
R^{2′} is H, halogen, C₁₋₆ alkyl optionally substituted by one or more halogen atoms, C₂₋₆ alkenyl optionally substituted by one or more halogen atoms, C₂₋₆ alkynyl optionally substituted by one or more halogen atoms, NH₂, NH(C₁₋₆ alkanoyl optionally substituted by one or more halogen atoms), NH(C₁₋₆ alkoxycarbonyl optionally substituted by one or more halogen atoms), N(C₁₋₆ alkoxycarbonyl optionally substituted by one or more halogen atoms)₂, NH(C₁₋₆ alkyl optionally substituted by one or more halogen atoms), N(C₁₋₆ alkyl optionally substituted by one or more halogen atoms)₂, NHCONH(C₁₋₆ alkyl optionally substituted by one or more halogen atoms), N-pyrrolyl, NHCONH(phenyl optionally substituted by one or more halogen atoms), N═CH(phenyl optionally substituted by one or more halogen atoms), OH, C₁₋₆ alkoxy optionally substituted by one or more halogen atoms, SH or S(O)ₘ (C₁₋₆ alkyl wherein C₁₋₆ alkyl is optionally substituted by one or more halogen atoms);
R³′, R^{4′}, R^{5′}, R^{6′} and R^{7′} are each independently H, halogen, nitro, C₁₋₆ alkoxycarbonyl optionally substituted by one or more halogen atoms, C₁₋₆ alkyl optionally substituted by one or more halogen atoms, CN, C₁₋₆ alkanoyl optionally substituted by one or more halogen atoms, CONH₂, CSNH₂, C₁₋₆ alkoxy optionally substituted by one or more halogen atoms, S(O)ₘ (C₁₋₆ alkyl wherein C₁₋₆ alkyl is optionally substituted by one or more halogen atoms) or SF₅;
m is 0, 1 or 2;
with the proviso that when
R^{1′} is H or C₁₋₆ alkyl,
R^{2′} is H, halogen, NH₂, NH(C₁₋₆ alkanoyl optionally substituted by one or more halogen atoms), NH(C₁₋₆ alkoxycarbonyl optionally substituted by one or more halogen atoms), N(C₁₋₆ alkoxycarbonyl optionally substituted by one or more halogen atoms)₂, NH(C₁₋₆ alkyl optionally substituted by one or more halogen atoms), NHCONH(C₁₋₆ alkyl optionally substituted by one or more halogen atoms), NHCONH(phenyl optionally substituted by one or more halogen atoms),
and R^{3′}, R^{4′}, R^{5′}, R^{6′} and R^{7′} are as defined for a compound of formula (I)
then Ar is not a heteroaryl group “A”;
or a pharmaceutically- or veterinarily-acceptable salt thereof.

4. The composition according to claim 3, wherein the composition further comprises a suitable pharmaceutical or veterinary carrier.

5. The composition according to claim 3, wherein the bis-aryl compound is of formula (I).

6. The composition according to claim 5, wherein R¹ is R¹ is CN, optionally substituted phenyl, optionally substituted C₁₋₆ alkyl, or C₁₋₆ alkoxycarbonyl.

7. The composition according to claim 5, wherein R² is a group of formula II where R⁷ is H, tri(C₁₋₆ alkyl)silyl, C₁₋₆ alkyl optionally substituted by one or more halogen, OH or C₁₋₆ alkoxy, or R⁷ is C₁₋₆ alkoxycarbonyl, phenyl, a 5- or 6-membered ring heterocycle, or halogen,
or R² is a group of formula III in which either R⁸, R⁹, and R¹⁰ are each H,
or a group of formula III in which two of R⁸, R⁹ and R¹⁰ are halogen and the other is H, CN, phenyl optionally substituted by one or more halogen or C₁₋₆ alkyl optionally substituted by one or more halogen,
or a group of formula III in which R⁸, R⁹ and R¹⁰ are each independently F, Cl, Br or I,
or a group of formula III in which R⁸ is H or C₁₋₆ alkyl optionally substituted by one or more halogen, OH or C₁₋₆ alkoxy, and R⁹ and R¹⁰ are both halogen,
or a group of formula III in which R⁸ is H and one of R⁹ and R¹⁰ is halogen and the other is C₁₋₆ alkyl optionally substituted by one or more halogen, OH or C₁₋₆ alkoxy,
or a group of formula III in which R⁸ is H and one of R⁹ and R¹⁰ is H and the other is CN or C₁₋₆ alkyl optionally substituted by one or more halogen, OH or C₁₋₆ alkoxy,
or a group of formula III in which R⁸ is H and R⁹ and R¹⁰ are C₁₋₆ alkyl optionally substituted by one or more halogen, OH or C₁₋₆ alkoxy,
or a group of formula III in which R⁸ is C₁₋₆ alkyl optionally substituted by one or more halogen, OH or C₁₋₆ alkoxy and R⁹ and R¹⁰ are both H,
or a group of formula IV.

8. The composition according to claim 5, wherein R³ is H, C₁₋₆ alkyl NH₂, NH(C₁₋₆ alkanoyl), NH(C₁₋₆ alkoxycarbonyl), N(C₁₋₆ alkoxycarbonyl)₂, N(C₁₋₆ alkyl)₂, N-pyrrolyl, halogen or S(O)ₙ (C₁₋₆ alkyl optionally substituted by one or more halogen) where n is 0, 1 or 2.

9. The composition according to claim 5, wherein R⁴ and R⁶ are each halogen.

10. The composition according to claim 5 wherein R⁵ is C₁₋₆ alkyl optionally substituted by one or more halogen, C₁₋₆ alkoxy optionally substituted by one or more halogen, C₁₋₆ alkylthio optionally substituted by one or more halogen, SF₅ or halogen.

11. The composition according to claim 5 wherein the compound of formula (I) is selected from:
3-cyano-1-(2,6-dichloro-4-trifluoromethylphenyl)-4-ethynylpyrazole;
3-cyano-1-(2,6-dichloro-4-trifluoromethoxyphenyl)-4-ethynylpyrazole;
3-cyano-1-(2,6-dichloro-4-trifluoromethylsulphenylphenyl)-4-ethynylpyrazole;
4-(2-bromo-1,2-dichloroethenyl)-3-cyano-1-(2,6-dichloro-4-trifluoromethoxyphenyl)-pyrazole;
3-cyano-1-(2,6-dichloro-4-trifluoromethoxyhenyl)-4-tribromoethenylpyrazole;
4-(2,2-dibromoethenyl)-3-cyano-1-(2,6-dichloro-4-trifluoromethylphenyl)-pyrazole;
3-cyano-4-(2,2-dichloroethenyl)-1-(2,6-dichloro-4-trifluoromethylphenyl)pyrazole, 3-cyano-1-(2,6-dichloro-4-trifluoromethylphenyl)-4-(2,2-difluoroethenyl)pyrazole;
3-cyano1-(2,6-dichloro-4-trifluoromethylphenyl)-4-tribromoethenylpyrazole;
3-cyano-1-(2,6-dichloro-4-trifluoromethylphenyl)-4-trichloroethenylpyrazole;
4-(2-bromo-1,2-dichloroethenyl)-3-cyano-1-(2,6-dichloro-4-trifluoromethylphenyl)-pyrazole;
4-(2-chloro-1,2-dibromoethenyl)-3-cyano-1-(2,6-dichloro-4-trifluoromethylphenyl)-pyrazole;
3-cyano-1-(2,6-dichloro-4-trifluoromethylphenyl)-4-(1-methyl-2,2-dibromoeth enyl)pyrazole;
3-cyano1-(2,6-dichloro-4-trifiluoromethylphenyl)-4-(1-methyl-2,2-difluoroet henyl)pyrazole;
1-(2,6-dichloro-4-trifluoromethylphenyl)-4-ethynyl-3-trifluoromethylpyrazole;
4-(2-bromo-1,2-dichloroethenyl)-1-(2,6-dichloro-4-trifluoromethylphenyl)-3-trifluoromethylpyrazole; and
1-(2,6-dichloro-4-trifluoromethylphenyl)-4-ethynyl-3-methylpyrazole, or salt thereof.

12. The composition according to claim 3, wherein the bis-aryl compound is of formula (I′).

13. The composition according to claim 12 wherein R^{1′} is CH₃,
R^{2′} is H or NH₂,
R^{3′} is halogen,
R^{5′} is H, C₁₋₆ alkyl optionally substituted by one or more halogen atoms, C₁₋₆ alkoxy optionally substituted by one or more halogen atoms or SF₅,
R^{7′} is halogen,
and Ar is phenyl, 3,4-methylenedioxyphenyl or naphth-1-yl, each optionally substituted by one or (independently) more halogen atoms, C₁₋₆ alkyl optionally substituted by one or more halogen atoms, C₁₋₆ alkoxy optionally substituted by one or more halogen atoms, C₁₋₆ alkoxycarbonyl optionally substituted by one or more halogen atoms, NO₂, NH₂, CN or S(O)ₘ (C₁₋₆ alkyl wherein C₁₋₆ alkyl is optionally substituted by one or more halogen atoms),
and pharmaceutically or veterinarily acceptable salts thereof.

14. The composition according to claim 12 wherein R³ is Cl.

15. The composition according to claim 12 wherein R^{5′} is H, CH₃, CF₃, OCF₃ or SF₅.

16. The composition according to claim 12 wherein R^{7′} is Cl.

17. The composition according to claim 12 wherein Ar is 4-methylphenyl, phenyl, 2-n-butylphenyl, 3-nitrophenyl, 4-bromophenyl, 4-chlorophenyl, 4-fluorophenyl, 3,5-dichlorophenyl, 2,3-dichlorophenyl, 3-chloro-4-fluorophenyl, 3-methoxycarbonylphenyl, 3-aminophenyl, 4-methoxyphenyl, 3,4-methylenedioxyphenyl, 3,4-dimethoxyphenyl, 3-fluorophenyl, 3-chlorophenyl, 2-fluorophenyl, 2-methoxyphenyl, 2-methylphenyl, 2-trifluoromethylphenyl, 2-chlorophenyl, or naphth-1-yl.

18. The composition according to claim 12 wherein Ar is 2-fluorophenyl, 2-methylphenyl, 2-n-butylphenyl, or 2,3-dichlorophenyl.

19. A composition comprising a combination of a synergistic amount of a bis-aryl compound of formula (V) and selamectin, wherein the compound of formula (V) is represented by: 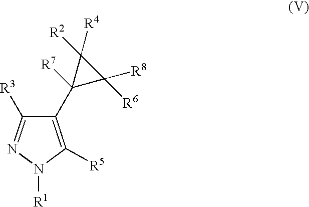
or a pharmaceutically, veterinarily or agriculturally acceptable salt thereof, or a pharmaceutically, veterinarily or agriculturally acceptable solvate of either entity, wherein
R¹ is 2,4,6-trisubstituted phenyl wherein the 2- and 6-substituents are each independently selected from halo and the 4-substituent is selected from C₁ to C₄ alkyl optionally substituted with one or more halo, C₁ to C₄ alkoxy optionally substituted with one or more halo, S(O)ₙC, to C₄ alkyl optionally substituted with one or more halo, halo and pentafluorothio;
or 3,5-disubstituted pyridin-2-yl wherein the 3 substituent is halo and the 5-substituent is selected from C₁ to C₄ alkyl optionally substituted with one or more halo, C₁ to C₄ alkoxy optionally substituted with one or more halo, S(O)ₙC₁ to C₄ alkyl optionally substituted with one or more halo, and pentafluorothio;
R³ is C₁ to C₄ alkyl optionally substituted with hydroxy or with one or more halo;
cyano, C, to C₅ alkanoyl or phenyl;
R⁵ is hydrogen, C₁ to C₄ alkyl, amino or halo;
R² and R⁴ are each independently selected from hydrogen, C₁ to C₄ alkyl, fluoro, chloro and bromo or, together with the carbon atom to which they are attached, form a C₃ to C₆ cycloalkyl group;
R⁶ and R⁸ are each independently selected from hydrogen, C, to C₄ alkyl, fluoro, chloro and bromo;
or, when R² and R⁴ do not form part of a cycloalkyl group, R² and R⁶, together with the carbon atoms to which they are attached, may form a C₅ to C₇ cycloalkyl group;
R⁷ is hydrogen, C₁ to C₄ alkyl optionally substituted with one or more halo, or C₁ to C₄ alkoxy;
and n is 0, 1 or 2.

20. The composition according to claim 19 wherein R¹ is 2,6-dichloro-4-trifluoromethylphenyl, 2,6-dichloro-4-pentafluorothiophenyl, 2,4,6-trichlorophenyl or 3-chloro-5-trifluoromethylpyridin-2-yl; R³ is methyl, ethyl, prop-2-yl, 1-hydroxyethyl, 2-hydroxyprop-2-yl, difluoromethyl, dichloromethyl, trifluoromethyl, cyano, formyl, acetyl or phenyl; R⁵ is hydrogen, methyl, amino or chloro; R² and R⁴ are each independently selected from hydrogen, methyl, fluoro, chloro and bromo or, together with the carbon atom to which they are attached, form a cyclopropyl, cyclobutyl or cyclopentyl group; R⁶ and R⁸ are each independently selected from hydrogen, methyl, chloro and bromo; or, when R² and R⁴ do not form part of a cycloalkyl group, R² and R⁸, together with the carbon atoms to which they are attached, may form a cyclopentane or cyclohexane group; and R⁷ is hydrogen, methyl, ethyl, trifluoromethyl, chlorodifluoromethyl, pentafluoroethyl, heptafluoropropyl or methoxy.

21. The composition according to claim 20 wherein R¹ is 2,6-dichloro-4-trifluoromethylphenyl, 2,6-dichloro-4-pentafluorothiophenyl or 3-chloro-5 trifluoromethylpyridin-2-yl; R³ is cyano; R⁵ is hydrogen or amino; R² and R⁴ are the same and are hydrogen, chloro or bromo; R⁶ and R⁸ are hydrogen; and R⁷ is hydrogen, trifluoromethyl or chlorodifluoromethyl.

22. The composition according to claim 21 wherein the compound of formula (V) is selected from:
3-cyano-4-(2,2-dibromocyclopropyl)-1-(2,6-dichloro-4-trifluoromethylphenyl)pyrazole;
(−)-3-cyano-4-(2,2-dibromocyclopropyl)-1-(2,6-dichloro-4-trifluoromethylphenyl)pyrazole;
3-cyano-1-(2,6-dichloro-4-trifluoromethylphenyl)-4-(1-trifluoromethylcyclopropyl)pyrazole;
3-cyano-4-(2,2-dibromocyclopropyl)-1-(2,6-dichloro-4-pentafluorothiophenyl)pyrazole;
3-cyano-4-(2,2-dichlorocyclopropyl)-1-(2,6-dichloro-4-trifluoromethylphenyl)pyrazole;
4-(1-chlorodifluoromethylcyclopropyl)-3-cyano-1-(2,6-dichloro-4-trifluoromethylphenyl)pyrazole;
1-[(3-chloro-5-trifluoromethyl)pyridin-2-yl]-3-cyano-4-(2,2-dibromocyclopropyl)pyrazole;
5-amino-3-cyano-4-(2,2-dibromocyclopropyl)-1-(2,6-dichloro-4-trifluoromethylphenyl)pyrazole;
5-amino-3-cyano-4-(2,2-dibromocyclopropyl)-1-(2,6-dichloro-4-pentafluorothiophenyl)pyrazole;
5-amino-3-cyano-4-(2,2-dichlorocyclopropyl)-1-(2,6-dichloro-4-pentafluorothiophenyl)pyrazole; and
5-amino-3-cyano-1-(2,6-dichloro-4-trifluoromethylphenyl)-4-(1-trifluoromethylcyclopropyl)pyrazole.

23. A composition comprising a combination of a synergistic amount of a bis-aryl compound of formula (VI) and selemectin, wherein the bis-aryl compound is represented by: 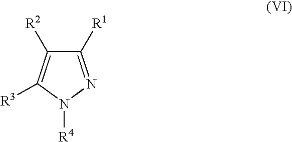
R¹ represents a cyano or nitro group, a halogen atom, or an acetyl or formyl group;
R² represents a group R⁵SO2, R⁵SO, or R⁵S in which R⁵ represents a straight- or branched-chain alkyl, alkenyl or alkynyl group containing up to 4 carbon atoms which may be unsubstituted or substituted by one or more halogen atoms which may be the same or different;
R³ represents a hydrogen atom or an amino group —NR⁶R⁷ wherein R⁶ and R⁷, which may be the same or different, each represent a hydrogen atom or a straight- or branched-chain alkyl, alkenylalkyl or alkynylalkyl group containing up to 5 carbon atoms, a formyl group, a straight- or branched-chain alkanoyl group (which contains from 2 to 5 carbon atoms and which may be optionally substituted by one or more halogen atoms) or R⁶ and R⁷ together with the nitrogen atom to which they are attached form a 5 or 6 membered cyclic imide, or represents a straight- or branched-chain alkoxycarbonyl group (which contains from 2 to 5 carbons atoms and is unsubstituted or substituted by one or more halogen atoms), or R³ represents a straight or branched-chain alkoxymethyleneamino group containing from 2 to 5 carbon atoms which may be unsubstituted or substituted on methylene by a straight or branched-chain alkyl group containing from 1 to 4 carbon atoms or represents a halogen atom; and R⁴ represents a phenyl group substituted in the 2-position by a fluorine, chlorine, bromine or iodine atom; in the 4-position by a straight- or branched-chain alkyl or alkoxy group containing from 1 to 4 carbon atoms, which may be unsubstituted or substituted by one or more halogen atoms which may be the same or different, or a chlorine or bromine atom; and optionally in the 6-position by a fluorine, chlorine, bromine or iodine atom, with the exclusion of the compound wherein R¹ represents cyano, R² represents methanesulphonyl, R³ represents amino and R⁴ represents 2,6-dichloro-4-trifluoromethylphenyl.

24. The composition according to claim 23 wherein R¹ is other than a formyl group and neither R⁶ nor R⁷ represents an alkenylalkyl or alkynylalkyl group.

25. The composition according to claim 23 wherein R² represents an alkylsulphonyl/sulphinyl/thio group which is optionally halogen substituted and contains from 1 to 4 carbon atoms, or an alkenyl- or alkynyl-sulphonyl/sulphinyl/thio group which is optionally halogen substituted and contains up to 4 carbon atoms, R³ represents the hydrogen atom, an amino or methylamino group and R¹ represents a halogen atom or a cyano or nitro group.

26. The compound according to claim 25 wherein R¹ represents a cyano or nitro group.

27. The composition according to claim 23 wherein R⁴ contains a trifluoromethyl or trifluoromethoxy group and R² represents an optionally halogenated alkylsulphonyl/sulphinyl/thio group containing from 1 to 4 carbon atoms.

28. The composition according to claim 24 wherein R⁴ contains a trifluoromethyl or trifluoromethoxy group and R² represents an optionally halogenated alkylsulphonyl/sulphinyl/thio group containing from 1 to 4 carbon atoms.

29. The composition according to claim 25 wherein R⁴ contains a trifluoromethyl or trifluoromethoxy group and R² represents an optionally halogenated alkylsulphonyl/sulphinyl/thio group containing from 1 to 4 carbon atoms.

30. The composition according to claim 26 wherein R⁴ contains a trifluoromethyl or trifluoromethoxy group and R² represents an optionally halogenated alkylsulphonyl/sulphinyl/thio group containing from 1 to 4 carbon atoms.

31. The composition according to claim 27 wherein R² represents a trifluoromethylthio, trifluoromethylsulphinyl or trifluoromethylsulphonyl group.

32. The composition according to claim 23 wherein R⁴ represents a 2,4,6-trichloro-, 2,6-dichloro-4-difluoromethoxy-, 2-chloro-4-trifluoromethyl-, 2-bromo-6-chloro-4-trifluoromethyl-, 2,6-dibromo-4-trifluoromethyl- or 2-bromo-4-trifluoromethyl-phenyl group.

33. The composition according to claim 24 wherein R⁴ represents a 2,4,6-trichloro-, 2,6-dichloro-4-difluoromethoxy-, 2-chloro-4-trifluoromethyl-, 2-bromo-6-chloro-4-trifluoromethyl-, 2,6-dibromo-4-trifluoromethyl- or 2-bromo-4-trifluoromethyl-phenyl group.

34. The composition according to claim 25 wherein R⁴ represents a 2,4,6-trichloro-, 2,6-dichloro-4-difluoromethoxy-, 2-chloro-4-trifluoromethyl-, 2-bromo-6-chloro-4-trifluoromethyl-, 2,6-dibromo-4-trifluoromethyl- or 2-bromo-4-trifluoromethyl-phenyl group.

35. The composition according to claim 26 wherein R⁴ represents a 2,4,6-trichloro-, 2,6-dichloro-4-difluoromethoxy-, 2-chloro-4-trifluoromethyl-, 2-bromo-6-chloro-4-trifluoromethyl-, 2,6-dibromo-4-trifluoromethyl- or 2-bromo-4-trifluoromethyl-phenyl group.

36. The composition according to claim 23 wherein R⁴ represents a 2,6-dichloro 4-trifluoromethyl- or 2,6-dichloro-4-trifluoromethoxy-phenyl group.

37. The composition according to claim 24 wherein R⁴ represents a 2,6-dichloro 4-trifluoromethyl- or 2,6-dichloro-4-trifluoromethoxy-phenyl group.

38. The composition according to claim 25 wherein R⁴ represents a 2,6-dichloro 4-trifluoromethyl- or 2,6-dichloro-4-trifluoromethoxy-phenyl group.

39. The composition according to claim 26 wherein R⁴ represents a 2,6-dichloro 4-trifluoromethyl- or 2,6-dichloro-4-trifluoromethoxy-phenyl group.

40. The composition according to claim 23 wherein the compound of formula (VI) is selected from the group consisting of:
5-amino-3-cyano-1-(2,6-dichloro-4-trifluoromethylphenyl)-4-trifluoromethylthiopyrazole;
5-amino-3-cyano-1-(2,6-dichloro-4-trifluoromethoxyphenyl)-4-trifluoromethylthiopyrazole;
5-amino-3-cyano-1-(2,6-dichloro-4-difluoromethoxyphenyl)-4-trifluoromethylthiopyrazole;
5-amino-1-(2-chloro-4-tri fluoromethylphenyl)-3-cyano-4-trifluoromethylthiopyrazole;
5-amino-3-cyano-1-(2,4,6-trichlorophenyl)-4-trifluoromethylthiopyrazole;
5-amino-3-cyano-1-(2,6-dibromo-4-trifluoromethylphenyl)-4-trifluoromethylthiopyrazole;
5-amino-4-trifluoromethylphenyl)-3-cyano-4-trifluoromethythiopyrazole;
5-amino-3-cyano-1-(2,6-dichloro-4-trifluoromethylphenyl)-4-difluoromethylthiopyrazole;
5-amino-3-cyano-1-(2,6-dichloro-4-trifluoromethylphenyl)-4-heptafluoropropylthiopyrazole;
5-amino-1-(2-bromo-6-chloro-4-trifluoromethylphenyl)-3-cyano-4-trifluoromethylthiopyrazole;
5-amino-3-cyano-1-(2,6-dichloro-4-trifluoromethylphenyl)-4-trichloromethylthiopyrazole;
5-amino-3-chloro-1-(2,6-dichloro-4-trifluoromethylphenyl)-4-trifluoromethylthiopyrazole;
5-amino-3-bromo-1-(2,6-dichloro-4-trifluoromethylphenyl)-4-trifluoromethylthiopyrazole;
5-amino-1-(2,6-dichloro-4-trifluoromethylphenyl)-3-fluoro-4-trifluoromethylthiopyrazole;
5-amino-4-chlorodifluoromethylthio-3-cyano-1-(2,6-dichloro-4-trifluoromethylphenyl)pyrazole;
5-chloro-3-cyano-1-(2,6-dichloro-4-trifluoromethylphenyl)-4-trifluoromethylthiopyrazole;
5-amino-3-chloro-1-(2,6-dichloro-4-trifluoromethylphenyl)-4-methanesulphonylpyrazole;
3-cyano-1-(2,6-dichloro-4-trifluoromethylphenyl)-5-ethoxymethyleneamino-4-trifluoromethylthiopyrazole;
3-cyano-1-(2,6-dichloro-4-trifluoromethylphenyl)-5-ethoxyethylideneamino-4-trifluoromethylthiopyrazole;
3-cyano-1-(2,6-dichloro-4-trifluoromethylphenyl)-5-ethoxymethyleneamino-4-methanesulphonylpyrazole;
5-acetamido-3-cyano-1-(2,6-dichloro-4-trifluoromethylphenyl)-4-trifluoromethylthiopyrazole;
3-cyano-1-(2,6-dichloro-4-trifluoromethylphenyl)-5-bis(propionyl)amino-4-trifluoromethylthiopyrazole;
3-cyano-1-(2,6-dichloro-4-trifluoromethylphenyl)-5-propionamido-4-trifluoromethylthiopyrazole;
5-acetamido-3-cyano-1-(2,6-dichloro-4-trifluoromethylphenyl)-4-methanesulphonylpyrazole;
3-cyano-1-(2,6-dichloro-4-trifluoromethylphenyl)-4-trifluoromethylthio-5-trimethylacetamidopyrazole;
3-cyano-1-(2,6-dichloro-4-trifluoromethylphenyl)-5-bis(methoxycarbonyl)amino-4-trifluoromethylthiopyrazole;
3-cyano-1-(2,6-dichloro-4-trifluoromethylphenyl)-5-bis(ethoxycarbonyl)amino-4-trifluoromethylthiopyrazole;
5-chloroacetamido-3-cyano-1-(2,6-dichloro-4-trifluoromethylphenyl-4-trifluoromethylthiopyrazole;
3-cyano-1-(2,6-dichloro-4-trifluoromethylphenyl)-5-bis(ethoxycarbonyl)amino-4-methanesulphonylpyrazole;
3-cyano-1-(2,6-dichloro-4-trifluoromethylphenyl)-4-methanesulphonyl-5-trimethylacetamidopyrazole;
3-cyano-1-(2,6-dichloro-4-trifluoromethylphenyl)-5-dimethylamino-4-trifluoromethylthiopyrazole;
3-cyano-1-(2,6-dichloro-4-trifluoromethylphenyl)-5-isopropylamino-4-trifluoromethylthiopyrazole;
3-cyano-1-(2,6-dichloro-4-trifluoromethylphenyl)-5-propylamino-4-trifluoromethylthiopyrazole;
3-cyano-1-(2,6-dichloro-4-trifluoromethylphenyl)-5-dipropylamino-4-trifluoromethylthiopyrazole;
3-cyano-1-(2,6-dichloro-4-trifluoromethylphenyl)-5-bis(propargyl)amino-4-trifluoromethylthiopyrazole;
3-cyano-1-(2,6-dichloro-4-trifluoromethylphenyl)-5-methylamino-4-methanesulphonylpyrazole;
5-bromo-3-cyano-1-(2,6-dichloro-4-trifluoroethylphenyl)-4-trifluoromethanesulphonylpyrazole;
5-bromo-3-cyano-1-(2,6-dichloro-4-trifluoromethylphenyl)-4-trifluoromethylthiopyrazole;
5-bromo-3-cyano-1-(2,6-dichloro-4-trifluoromethylphenyl)-4-methanesulphonylpyrazole;
5-amino-3-bromo-1-(2,6-dichloro-4-trifluoromethylphenyl)-4-methanesulphonylpyrazole;
3-bromo-1-(2,6-dichloro-4-trifluoromethylphenyl)-4-methanesulphonylpyrazole;
3-cyano-1-(2,6-dichloro-4-trifluoromethylphenyl)-4-trifluoromethylthiopyrazole;
3-cyano-1-(2,6-dichloro-4-trifluoromethylphenyl)-4-trifluoromethanesulphonylpyrazole;
3-cyano-1-(2,6-dichloro-4-trifluoromethoxyphenyl)-4-trifluoromethylthiopyrazole;
3-cyano-1-(2,6-dichloro-4-trifluoromethylphenyl)-4-methanesulphonylpyrazole;
3-cyano-1-(2,6-dichloro-4-trifluoromethylphenyl)-5-iodo-4-trifluoromethylthiopyrazole;
3-cyano-1-(2,6-dichloro-4-trifluoromethylphenyl)-5-iodo-4-trifluoromethanesulphonylpyrazole;
5-amino-1-(2,6-dichloro-4-trifluoromethylphenyl)-3-iodo-4-methanesulphonylpyrazole;
1-(2,6-dichloro-4-trifluoromethylphenyl)-3-iodo-4-methanesulphonylpyrazole;
1-(2,6-dichloro-4-trifluoromethylphenyl)-3-iodo-4-trifluoromethylthiopyrazole;
5-amino-3-cyano-1-(2,6-dichloro-4-trifluoromethylphenyl)-4-trifluoromethanesulphonylpyrazole;
5-amino-3-cyano-1-(2,6-dichloro-4-trifluoromethylphenyl)-4-trifluoromethylsulphinylpyrazole;
5-amino-3-cyano-1-(2,6-dichloro-4-trifluoromethoxyphenyl)-4-trifluoromethanesulphonylpyrazole;
5-amino-3-cyano-1-(2,6-dichloro-4-trifluoromethoxyphenyl)-4-trifluoromethylsulphinylpyrazole;
3-cyano-1-(2,6-dichloro-4-trifluoromethylphenyl)-4-trifluoromethylsulphinylpyrazole;
5-amino-3-cyano-1-(2,6-dichloro-4-trifluoromethylphenyl)-4-(1-methylprop-2-ynylsulphinyl)pyrazole;
5-amino-3-cyano-1-(2,6-dichloro-4-trifluoromethylphenyl)-4-methylsulphinylpyrazole;
5-amino-3-cyano-1-(2,6-dichloro-4-trifluoromethylphenyl)-4-isopropylsulphinylpyrazole;
5-amino-3-bromo-1-(2,6-dichloro-4-trifluoromethylphenyl)-4-trifluoromethylsulphinylpyrazole;
5-amino-4-tert-butanesulphonyl-3-cyano-1-(2,6-dichloro-4-trifluoromethylphenyl)pyrazole;
3-cyano-1-(2,6-dichloro-4-trifluoromethylphenyl)-5-propylamino-4-trifluoromethylsulphonylpyrazole;
5-acetamido-3-cyano-1-(2,6-dichloro-4-trifluoromethylphenyl)-4-trifluoromethanesulphonylpyrazole;
1-(2,6-dichloro-4-trifluoromethylphenyl)-4-methanesulphonyl-3-nitropyrazole;
5-amino-1-(2,6-dichloro-4-trifluoromethylphenyl)-4-methanesulphonyl-3-nitropyrazole;
1-(2,6-dichloro-4-trifluoromethylphenyl)-3-nitro-4-trifluoromethylsulphinylpyrazole;
5-amino-1-(2-bromo-6-chloro-4-trifluoromethylphenyl)-3-cyano-4-methanesulphonylpyrazole;
5-amino-1-(2,6-dichloro-4-trifluoromethoxyphenyl)-3-cyano-4-methanesulphonylpyrazole;
3-acetyl-5-amino-1-(2,6-dichloro-4-trifluoromethylphenyl)-4-methanesulphonylpyrazole;
5-amino-3-cyano-1-(2,6-dichloro-4-trifluoromethylphenyl)-4-methylthiopyrazole;
5-amino-3-cyano-1-(2,6-dichloro-4-trifluoromethylphenyl)-4-ethylthiopyrazole;
5-amino-3-cyano-1-(2,6-dichloro-4-trifluoromethylphenyl)-4-propylthiopyrazole;
5-amino-3-cyano-1-(2,6-dichloro-4-trifluoromethylphenyl)-4-isopropylthiopyrazole;
5-amino-3-cyano-1-(2,6-dichloro-4-trifluoromethylphenyl)-4-(2-methylpropylthio)pyrazole;
5-amino-3-cyano-1-(2,6-dichloro-4-trifluoromethylphenyl)-4-(1-methylpropylthio)pyrazole;
4-allylthio-5-amino-3-cyano-1-(2,6-dichloro-4-trifluoromethylphenyl)pyrazole;
5-amino-3-cyano-1-(2,6-dichloro-4-trifluoromethylphenyl)-4-(prop-2-ynylthio)pyrazole;
5-amino-3-cyano-1-(2,6-dichloro-4-trifluoromethylphenyl)-4-(1-methylprop-2-ynylthio)pyrazole;
5-amino-3-bromo-1-(2,6-dichloro-4-trifluoromethylphenyl)-4-methylthiopyrazole;
5-amino-3-cyano-1-(2,6-dichloro-4-trifluoromethylphenyl)-4-tert-butylthiopyrazole;
5-amino-3-bromo-1-(2,6-dichloro-4-trifluoromethylphenyl)-4-methylsulphinylpyrazole;
5-amino-3-cyano-1-(2,6-dichloro-4-trifluoromethylphenyl)-4-ethanesulphonylpyrazole;
3-cyano-1-(2,6-dichloro-4-trifluoromethylphenyl)-5-methylamino-4-trifluoromethylthiopyrazole;
3-cyano-1-(2,6-dichloro-4-trifluoromethylphenyl)-5-(n-ethoxycarbonyl-n-methyl)amino-4-trifluoromethylthiopyrazole;
3-cyano-1-(2,6-dichloro-4-trifluoromethylphenyl)-5-trifluoroacetamido-4-trifluoromethylthiopyrazole;
3-cyano-1-(2,6-dichloro-4-trifluoromethylphenyl)-5-(ethoxycarbonylamino)-4-trifluoromethylthiopyrazole;
3-acetyl-1-(2,6-dichloro-4-trifluoromethylphenyl)-4-trifluoromethylthiopyrazole;
1-(2,6-dichloro-4-trifluoromethylphenyl)-3-formyl-4-trifluoromethylthiopyrazole;
5-amino-1-(2,6-dichloro-4-trifluoromethylphenyl)-3-formyl-4-trifluoromethylthiopyrazole;
3-cyano-1-(2,6-dichloro-4-trifluoromethylphenyl)-5-fluoro-4-trifluoromethanesulphonylpyrazole;
5-amino-3-cyano-4-dichlorofluoromethylthio-1-(2,6-dichloro-4-trifluoromethylphenyl)pyrazole;
5-amino-3-chloro-1-(2,6-dichloro-4-trifluoromethylphenyl)-4-trifluoromethanesulphonylpyrazole;
5-amino-3-cyano-1-(2,6-dichloro-4-trifluoromethylphenyl)-4-pentafluoroethylthiopyrazole;
3-cyano-1-(2,6-dichloro-4-trifluoromethylphenyl)-5-dimethylamino-4-trifluoromethylsulphinylpyrazole;
3-cyano-1-(2,6-dichloro-4-trifluoromethylphenyl)-5-iodo-4-trifluoromethylsulphinylpyrazole;
5-bromo-3-cyano-1-(2,6-dichloro-4-trifluoromethylphenyl)-4-trifluoromethylsulphinylpyrazole;
5-acetamido-3-cyano-1-(2,6-dichloro-4-trifluoromethylphenyl)-4-trifluoromethylsulphinylpyrazole;
3-cyano-1-(2,6-dichloro-4-trifluoromethylphenyl)-5-bis(ethoxycarbonyl)amino-4-trifluoromethanesulphonylpyrazole;
3-cyano-1-(2,6,dichloro-4-trifluoromethylphenyl)-5-ethoxycarbonylamino-4-trifluoromethanesulphonylpyrazole;
3-cyano-1-(2,6-dichloro-4-trifluoromethylphenyl)-5-ethoxymethyleneamino-4-trifluoromethanesulphonylpyrazole;
5-amino-4-(2-chloro-1,1,2-trifluoroethylthio)-3-cyano-1-(2,6-dichloro-4-trifluoromethylphenyl)pyrazole; and
3-cyano-1-(2,6-dichloro-4-trifluoromethylphenyl)-5-dimethylamino-4-trifluoromethanesulphonylpyrazole.

41. The composition according to claim 23 wherein the compound of formula (VI) is 5-amino-3-cyano-1-(2,6-dichloro-4-trifluoromethylphenyl)-4-trifluoromethylsulphinylpyrazole).

42. The composition comprising a combination of a synergistic amount of a bis-aryl compound of formula (VII) and selamectin, wherein the bis-aryl compound VII is represented by: 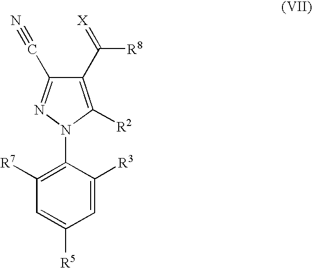
wherein
R² is NH₂, H, halogen, NH(C₁₋₆ alkyl optionally substituted with one or more halogen), NH(allyl optionally substituted with one or more halogen), NH(benzyl optionally substituted with one or more halogen), NHSCF₃, or R² is C₁₋₆ alkyl optionally substituted with one or more halogen;
R³, R⁵ and R⁷ are each independently H, halogen, SF₅, C₁₋₆ alkyl optionally substituted with one or more halogen, C₁₋₆ alkoxy optionally substituted with one or more halogen, or S(O)ₙ(C₁₋₆ alkyl wherein C₁₋₆ alkyl is optionally substituted with one or more halogen) wherein n is 0, 1 or 2;
X is O or NOY;
Y is H or C₁₋₆ alkyl optionally substituted with one or more halogen; R is H, C₁₋₈ alkyl optionally substituted with one or more halogen, or
C₃₋₈ cycloalkyl optionally substituted by one or more halogen, or by one or more C₁₋₄ alkyl optionally substituted with one or more halogen, or by C₃₋₈ cycloalkylcarbonyl; with the proviso that the compound for formula (VII) is not 3-cyano-4-formyl-1-phenylpyrazole; or a pharmaceutically- or veterinarily-acceptable acid addition salt thereof.

43. The composition according to claim 42 wherein R² is NH₂, H, halogen, NH(C₁₋₁₆ alkyl optionally substituted with one or more halogen), NH(allyl optionally substituted with one or more halogen), NH(benzyl optionally substituted with one or more halogen), NHSCF₃, or R₂ is C₁₋₆ alkyl optionally substituted with one or more halogen.

44. The composition according to claim 42 wherein R₃ and R₇ are halogen.

45. The composition according to claim 42 wherein R⁵ is SF₅, C₁₋₆ alkyl optionally substituted with one or more halogen or C₁₋₆ alkoxy optionally substituted with one or more halogen.

46. The composition according to claim 42 wherein X is O or NOY where Y is H or C₁₋₆ alkyl.

47. The composition according to claim 42 wherein R⁸ is H, C₁₋₈ alkyl optionally substituted with one or more halogen, or C₃₋₈ cycloalkyl optionally substituted by one or more C₁₋₄ alkyl.

48. The composition according to claim 42 wherein R² is H, NHCH₃, NHCH₂Ph, NHCH₂CH═CH₂, NHCH(CH₃)₂, NHSCF₃ or NH₂.

49. The composition according to claim 42 wherein R³ and R⁷ are Cl.

50. The composition according to claim 42 wherein R⁵ is OCF₃, CF₃ or SF₅.

51. The composition according to claim 42 wherein X is O, NOH or NOCH₃.

52. The composition according to claim 42 wherein R₈ is H, CH₃, CF₃, CH(CH₃)₂, cyclobutyl, 2,2,3,3-tetramethylcyclopropyl, C₂F₅, C₃F₇, C₂H₅, CF₂Cl, CCl₂F or cyclopropyl.

53. The composition according to claim 42 wherein X is 0.

54. The composition according to claim 42 wherein the compound of formula (VII) is selected from:
4-acetyl-5-amino-3-cyano-1-(2,6-dichloro-4-trifluoromethylphenyl)pyrazole;
4-acetyl-3-cyano-1-(2,6-dichloro-4-trifluoromethylphenyl)pyrazole;
5-amino-3-cyano-1-(2,6-dichloro-4-trifluoromethylphenyl)-4-(1-oximinoethyl)pyrazole;
5-amino-3-cyano-1-(2,6-dichloro-4-trifluoromethylphenyl)-4-(1-methoximinoethyl)pyrazole;
S-amino-3-cyano-1-(2,6-dichloro-4-trifluoromethylphenyl)-4-methoximinomethylpyrazole;
5-amino-3-cyano-1-(2,6-dichloro-4-trifluoromethylphenyl)-4-trifluoroacetylpyrazole;
3-cyano-1-(2,6-dichloro-4-trifluoromethylphenyl)-4-oximinomethylpyrazole;
3-cyano-1-(2,6-dichloro-4-trifluoromethylphenyl)-4-(2-methylpropanoyl)pyrazole;
3-cyano-4-cyclopropylcarbonyl-1-(2,6-dichloro-4-trifluoromethylphenyl)pyrazole;
4-acetyl-5-amino-3-cyano-1-(2,6-dichloro-4-sulphurpentafluorophenyl)pyrazole;
4-acetyl-3-cyano-1-(2,6-dichloro-4-sulphurpentafluorophenyl)pyrazole;
4-acetyl-5-amino-3-cyano-1-(2,6-dichloro-4-trifluoromethoxyphenyl)pyrazole;
4-acetyl-3-cyano-1-(2,6-dichloro-4-trifluoromethoxyphenyl)pyrazole;
3-cyano-1-(2,6-dichloro-4-trifluoromethylphenyl)-5-methylamino-4-trifluoroacetylpyrazole;
3-cyano-1-(2,6-dichloro-4-trifluoromethylphenyl)-5-(prop-2-enylamino)-4trifluoroacetylpyrazole;
3-cyano-1-(2-(2,6-dichloro-4-trifluoromethylphenyl)-5-phenylmethylamino-4trifluoroacetylpyrazole; and
3-cyano-1-(2,6-dichloro-4-trifluoromethylphenyl)-5-(prop-2-ylamino)-4-trifluoroacetylpyrazole.

55. The composition of claim 1, wherein the weight ratio of selemectin to fipronil is in the range of 1 to 100.

56. The composition of claim 1, wherein the weight ratio of selemectin to fipronil is in the range of 1 to 10.
